(19) United States
(12) Patent Application Publication
(10) Pub. No.: US 2022/0062177 A1
(43) Pub. Date: Mar. 3, 2022

(54) EXTENDED RELEASE FORMULATIONS OF HUMAN CHORIONIC GONADOTROPIN (HCG)

(71) Applicants: InnoCore Technologies Holding B.V., Groningen (NL); MHB Labs, Inc., Lexington, MA (US)

(72) Inventors: Johan ZUIDEMA, Aduard (NL); Rob STEENDAM, Groningen (NL); Joana Catarina Ribeiro ARAUJO, Zwolle (NL); Ravi KACKER, Lexington, MA (US); Abraham MORGENTALER, Brookline, MA (US)

(73) Assignees: InnoCore Technologies Holding B.V., Groningen (NL); MHB Labs, Inc., Lexington, MA (US)

(21) Appl. No.: 17/281,902

(22) PCT Filed: Oct. 2, 2019
(86) PCT No.: PCT/NL2019/050660

§ 371 Date: Mar. 31, 2021

Related U.S. Application Data
(60) Provisional application No. 62/740,145, filed on Oct. 2, 2018.

(51) Int. Cl. A61K 9/16; A61K 38/24; A61K 9/00

(52) U.S. Cl. CPC A61K 9/1641; A61K 9/0002; A61K 38/24

(57) ABSTRACT

The present disclosure relates to extended release dosage forms of human chorionic gonadotropin (hCG) or derivatives or isoforms thereof. The extended release hCG dosage forms comprise biodegradable polymer microspheres and provide for extended release of hCG over a desired time period.

This application is the U.S. National Stage of International Application No. PCT/NL2019/050660, filed Oct. 2, 2019, which claims benefit of priority to U.S. Provisional Application No. 62/740,145, filed Oct. 2, 2018, the entire contents of which are incorporated herein by reference.

### I. BACKGROUND

Human chorionic gonadotropin (hCG) is a hormone produced by the syncytiotrophoblast cells of the human placenta and gonads. hCG interacts with the luteinizing hormone/choriogonadotropin receptor (LHCGR) (also known as lutropin/choriogonadotropin receptor (LCGR) or luteinizing hormone receptor (LHR) found in the gonads of both genders. The action of hCG is similar to that of pituitary luteinizing hormone (LH), in that: both hormones stimulate production of testosterone and other steroid hormones by the Leydig cells of the testis and both hormones stimulate production of progesterone by the corpus luteum of the ovary.

During fetal development, hCG produced by the placenta stimulates the fetal testes to produce androgens, which are important to normal male sexual development. In the adult, administration of exogenous hCG stimulates testosterone production from the Leydig cells of the testes. For men with hypogonadotrophic hypogonadism, exogenously administered hCG can stimulate the testicular Leydig cells and restore normal testosterone production. Administration of hCG may also stimulate testicular descent in boys with cryptorchidism when no anatomical impediment to descent is present.

In the female, hCG produced by the placenta stimulates the ovary and promotes the maintenance of the corpus luteum during the beginning of pregnancy. This allows the corpus luteum to secrete the hormone progesterone during the first trimester. Progesterone enriches the uterus with a thick lining of blood vessels and capillaries so that it can sustain the growing fetus.

During the normal menstrual cycle, LH participates with FSH in the development and maturation of the normal ovarian follicle, and the mid-cycle LH surge triggers ovulation. In adult females, clinical administration of exogenous hCG can substitute for a LH surge. For women undergoing in vitro fertilization, hCG is extensively used parenterally for final maturation induction. In the presence of one or more mature ovarian follicles, ovulation can be triggered by the administration of hCG. In addition, hCG is sometimes used to enhance the production of progesterone for clinical purposes during treatment for infertility.

As the most abundant biological source is women who are presently pregnant, some organizations collect urine from pregnant women to extract hCG for pharmaceutical use, in dosage forms marketed under the trade names Novarel® and Pregnyl®. Recombinant hCG is produced through Chinese hamster ovary (CHO) cells and commercially available in a dosage form marketed under the trade name Ovidrel®.

Current commercially available dosage forms of hCG are limited to intramuscular (IM) or subcutaneous (SC) injectable forms which raise serum hCG levels to therapeutic levels over a short period of time. Frequent injections are required for several clinical applications where sustained dosing of hCG is needed. These applications include but are not limited to treatment of hypogonadotrophic hypogonadism and stimulation of progesterone production for female fertility. There is a need in the art for extended release dosage forms of hCG. The present invention satisfies this need.

### II. SUMMARY

The present disclosure relates to the long felt need in the art for extended release human chorionic gonadotropin (hCG) formulations. In particular, the present disclosure is directed to hCG dosage forms having extended release profiles. In some embodiments, the hCG dosage forms exhibit release profiles of between about 1 week and about 2 months. In other embodiments, the hCG dosage forms described herein can have extended release profiles between about 1 week and about 6 months.

In some aspects, the extended release hCG dosage form comprises hCG encapsulated in a microsphere. In further aspects, the microsphere is formed by a copolymer. In still further aspects, the copolymer is a block copolymer or a multi-block copolymer. In such aspects, the block copolymer may comprise, or alternatively consists essentially of, polyethylene glycol (PEG) or a PEG-containing polymeric block and one or more other polymeric blocks.

hCG extended release formulations described herein may be useful in a variety of treatments relating to hormone therapy, including but not limited to treatment for infertility and pituitary gland disorders. Thus, further aspects of the disclosure relate to methods of administering the extended release hCG formulations described herein, as well as methods of treatment employing the extended release hCG formulations described herein. Such methods include treatments for fertility and pituitary gland defects. Further methods disclosed herein include treatment of breast cancer. In some embodiments, the treatment is for existing breast cancer in nulliparous women. In some embodiments, the women are about age 25 or younger.

In another embodiment, encompassed are methods of administering the described extended release hCG dosage forms according to a regimen that achieves or approximates the theoretical release profile of FIG. 1.

The inventions described and claimed herein have many attributes and embodiments including, but not limited to, those set forth or described or referenced in this Summary. It is not intended to be all-inclusive and the inventions described and claimed herein are not limited to or by the features or embodiments identified in this Summary, which is included for purposes of illustration only and not restriction. Additional embodiments may be disclosed in the Description of the Figures and Detailed Description below.

### III. BRIEF DESCRIPTION OF THE DRAWINGS

FIG. 1 Exemplary theoretical release profile for an hCG extended release formulation.

FIG. 2 Cumulative release profile (μg released) of hCG extended release formulations comprising hCG microspheres, showing the total amount of hCG released over a period of 14 days; panel A depicts the cumulative release of each individual formulation, and panel B depicts the average cumulative release of each formulation.

FIG. 3 Release profile of hCG extended release formulations comprising hCG microspheres normalized for the total content of hCG (% released) for the hCG microspheres. The release profile shows the total amount of hCG released over a period of 14 days; panel A depicts the cumulative release of each individual formulation, and panel B depicts the average cumulative release of each formulation.

FIG. 4 Actual in vitro release profile (% release) over a period of 14 days calculated for formulation 11, which is an hCG extended release formulation comprising hCG microspheres.

FIG. 5 Predicted release profile of formulation 11 based on regimented administration of 3 mg every 7 days with a Cₘᵢₙ of 18 ng/ml.

FIG. 6 Predicted release profile of formulation 11 based on regimented administration of 3 mg every 14 days with a Cₘᵢₙ of 1.7 ng/ml.

FIG. 7 Predicted release profile of formulation 11 based on regimented administration of 3 mg every 28 days with a Cₘᵢₙ of 0.07 ng/ml.

FIG. 8 Predicted release profile of formulation 11 based on regimented administration of 250 μg every 7 days with a t^{1/2} of 36 hours and a Cₘᵢₙ of 3 ng/ml.

FIG. 9 Predicted release profile of formulation 11 based on regimented administration of 250 μg every 14 days with a t^{1/2} of 36 hours and a Cₘᵢₙ of 0.3 ng/ml.

FIG. 10 Cumulative release profile (μg released) of hCG extended release formulations comprising hCG microspheres, demonstrating the total amount of hCG released over a period of 50 days; panel A depicts the average cumulative release of each individual formulation, and panel B depicts the normalized release profile (% release) of each formulation.

FIG. 11 HPLC chromatogram demonstrating the purity of hCG upon concentration thereof.

FIG. 12 Gel analysis of the molecular weight of Ovidrel® as compared to Dong-A hCG.

FIG. 13 Purity analysis of the hCG from round 1 in vitro release.

FIG. 14 Cumulative release profile of extended release formulations comprising hCG microspheres (run in three replicates for each formulation), demonstrating the total amount of hCG released over a period of 40 days: panel A depicts the cumulative release profile (μg released) of each replicate, panel B depicts the normalized profile (% based on high performance size-exclusion chromatography), panel C depicts the normalized release profile (% release) of each formulation.

FIG. 15 Average release profiles of the replicates depicted in FIG. 14.

FIG. 16 Cumulative release profile of for three replicate runs for extended release formulation 695-01-0034 comprising hCG microspheres, demonstrating the total amount of hCG released over a period of 40 days: panel A depicts the cumulative release profile (μg released) of each replicate, panel B depicts the normalized profile (% based on high performance size-exclusion chromatography), panel C depicts the normalized release profile (% release) of each formulation.

FIG. 17 RP-UPLC chromatogram of concentrated hCG solution.

FIG. 18 SEM photograph of hCG extended release microspheres composed of a blend of SynBiosys 50CP10C20-LL40 and 30CP30C40-LL40.

FIG. 19 Cumulative release profiles of hCG extended release formulations comprising hCG microspheres composed of blends of SynBiosys 50CP10C20-LL40 and 30CP30C40-LL40. The release profiles (average of three replicates for each formulation) are normalized for the total content of hCG (% released) of each formulation and show the total amount of hCG released over a period of 60 days.

FIG. 20 Cumulative release profile of hCG extended release formulations comprising hCG microspheres composed of a 33/66% w/w blend of SynBiosys 50CP10C20-LL40 and 30CP30C40-LL40 (JA16043). The release profile (average of three replicates for each formulation) is normalized for the total content of hCG (% released) of the formulation and shows the total amount of hCG released and the % intact hCG (relative to the total amount) over a period of 35 days.

FIG. 21 SEM photograph of hCG extended release microspheres composed of 20[PCL-PEG3000-PCL]-b-[PDO] (batch nr JA16101).

FIG. 22 Cumulative release profile of hCG extended release formulations composed of SynBiosys 20[PCL-PEG3000-PCL]-b-[PDO] (JA16101 and JA16102). The release profiles (average of three replicates for each formulation) are normalized for the total content of hCG (% released) of each formulation and show the total amount of hCG released and the % intact hCG (relative to the total amount) over a period of 70 days.

FIG. 23 Serum hCG levels (panel A) and serum testosterone levels (panel B) in control group monkeys receiving daily hCG injections.

FIG. 24 Serum r-hCG and testosterone levels in monkeys treated with hCG extended release microspheres representing total doses of 200, 600 and 1200 μg hCG: panel A depicts the serum r-hCG levels for the first 48 hours, panel B depicts the serum r-hCG levels for the complete 28 days duration of the study and panel C depicts the serum testosterone levels for the complete 28 days duration of the study.

FIG. 25 Serum r-hCG and testosterone levels in monkeys treated with hCG extended release microspheres representing total doses of 200 μg (panel A), 600 μg (panel B) and 1200 μg hCG (panel C).

FIG. 26 SEC-UPLC chromatogram of rhCG and its potential degradation products.

FIG. 27 Cumulative release profile of Ovidrel hCG extended release formulations composed of SynBiosys 20[PCL-PEG3000-PCL]-b-[PDO]. The release profiles (average of three replicates for each formulation) are normalized for the total content of hCG (% released) of each formulation and show the total amount of hCG released over a period of 49 days as measured with the optimized in vitro dissolution method.

FIG. 28 Percent of hCG-induced progesterone produced relative to progesterone produced by Ovidrel. A) hCG released from microsphere lots at 2 hrs, 23 and 37 days; B) hCG extracted from microsphere (AMD-18-022-1) or spiked (AMD-18-022-4) in extraction buffer and C) Average percent progesterone produced by hCG released from lots MS18-037, MS18-038 and SR18-031 as a function of time, relative to Ovidrel.

FIG. 29 Results of 2-AB glycan mapping of hCG extracted from hCG extended release microspheres.

### IV. DETAILED DESCRIPTION

The present invention is directed to extended release hCG dosage forms. There has been a long felt need in the art for such dosage forms as presently all of the commercially available hCG dosage forms are liquids for intramuscular or subcutaneous injection. Further, all of the current commercially available hCG dosage forms have an immediate release profile, so that serum levels are not sustained and limited by the half-life of hCG, about 36 hours. This means that patients need to take frequent injections to obtain the desired hCG therapeutic level over a period of time, which can be inconvenient and therefore result in poor patient compliance or lead patients and providers to choose less effective treatments.

In some aspects, the extended release hCG dosage form comprises hCG or a derivative or isoform thereof encapsulated in a microsphere. In further aspects, the microsphere is formed by a copolymer. In still further aspects, the copolymer is a block copolymer or a multi-block copolymer. In such aspects, the block copolymer may comprise, or alternatively consists essentially of, polyethylene glycol (PEG) or a PEG-containing polymeric block, and one or more other polymers.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

### A. Human Chorionic Gonadotropin

As used herein the term “human chorionic gonadotropin” or “hCG” refers to a specific glycoprotein associated with this name and any other molecules that have analogous biological function that share at least about 80% amino acid sequence identity with naturally occurring hCG or an isoform thereof. hCG derivatives and isoforms can also be utilized in the compositions and methods described herein. In other embodiments, hCG variants having at least about 85%, at least about 90%, at least about 91%, at least about 92%, at least about 93%, at least about 94%, at least about 95%, at least about 96%, at least about 97%, at least about 98%, at least about 99%, or about 100% amino acid sequence identity with naturally occurring hCG can be used in the compositions of the invention.

In nature, hCG is produced by the syncytiotrophoblast in the placenta following implantation and in small amounts by the gonads. It is an analog of LH, which is produced in the pituitary gland of males and females of all ages. hCG may also be produced synthetically and is commercially available. hCG as used herein may refer to recombinant versions of the glycoprotein such as, but not limited to, Ovidrel®, Dong-A's recombinant hCG [product code DA-3803], another recombinant hCG that demonstrates bioequivalence to either of these products, or generic versions thereof. See, e.g., Seo et al., BioDrugs, 1; 25(2):115-27 (2011). In some embodiments, such a recombinant hCG is produced by an animal (e.g. a human or other mammal) cell line or a bacterial cell line. In some embodiments, such a recombinant hCG is between about 40 and about 60 kDa, e.g. at least or about 40 kDa, at least or about 41 kDa, at least or about 42 kDa, at least or about 43 kDa, at least or about 44 kDa, at least or about 45 kDa, at least or about 46 kDa, at least or about 47 kDa, at least or about 48 kDa, at least or about 49 kDa, at least or about 50 kDa, at least or about 51 kDa, at least or about 52 kDa, at least or about 53 kDa, at least or about 54 kDa, at least or about 55 kDa, at least or about 56 kDa, at least or about 57 kDa, at least or about 58 kDa, at least or about 59 kDa, or about 60 kDa. hCG as used herein may also refer to isolated naturally-produced hCG such as, but not limited to, urine-derived hCGs of Novarel®, Pregnyl®, another recombinant hCG that demonstrates bioequivalence to either of these products, or generic versions thereof.

hCG is a heterodimer of about 38 kDa comprising alpha and beta subunits. The alpha subunit has 92 amino acids and two N-linked carbohydrate chains. The beta subunit has 145 amino acids, with two N-linked and four O-linked carbohydrate chains. The individual subunits are held together by non-covalent interactions in the heterodimer, while the heterodimer and the alpha and beta subunits have little or no hydrophobic core. Disulfide linkages, five the in the alpha-subunit and six in the beta-subunit, stabilize the structures. There is a homologous placement of ten half cystines in the alpha subunit and the beta subunit contains twelve conserved half cystines. Banerjee et al., Indian J. of Exp. Biol., 40:434-447 (2002). Both of the subunits consist of a cystine-knot motif formed by three disulfide bridges and four polypeptide chains. In each subunit, three hairpin loops emerge from the central cystine knot. Glycoprotein hormones, such as hCG, are the most complex molecules with hormonal activity. Cahoreau et al., Front Endocrinol. (Lausanne), 6:26 (2015).

hCG heterogeneity: Numerous molecular forms of hCG are present in pregnancy serum, included dissociated or degraded molecules with no biological activity. Examples of known isoforms of hCG include intact hCG (“hCG”), nicked hCG (“hCGn”), hCG beta-subunit (“hCGβ”), nicked hCG beta-subunit (“hCGβn”), hCG beta core fragment (“hCGβcf”), and hCG alpha-subunit (“hCGα”).

### B. Copolymer Microspheres

As used herein, the term “microsphere” refers to a spherical or spheroid particle about 999 μm or less in diameter encapsulating an internal void in which may be loaded one or more therapeutic agents for drug delivery.

Aspects of the disclosure relate to microspheres formed by copolymers. In some embodiments, these copolymers may be selected based on the copolymer or a portion thereof having one or more of the following characteristics: (i) the polymer forms a lattice to allow the free flow of acid or the release of acid, (ii) the polymer protects the hCG active ingredient from the environment in which it is stored and/or into which the microsphere is released (e.g. temperature stability), (iii) the polymer is hydrophilic, (iv) the polymer degrades without impacting the purity of hCG, (v) the polymer is biodegradable, (vi) the polymer enables diffusion of the active ingredient (hCG), and/or (vii) the polymer will accommodate the hydrodynamic radius of hCG (such as that of Ovidrel® or Dong-A hCG).

The microsphere may release less than about 3% to about 40% of the hCG therein based on total weight of the microsphere, within about 24 hours.

It was unexpected that stable extended release formulations of hCG utilizing polymers as described herein could be made as hCG is known to degrade at elevated temperatures, such as body temperatures. It is also known to degrade in response to pH changes. Thus, prior to the present invention it was thought that the only way to successfully administer hCG to a patient or subject was via injection. The present invention details the surprising discovery that polymers can be used to form a stable extended release formulation of hCG, where the component hCG is not degraded by temperature or pH present in vivo.

Microspheres comprising hCG or a derivative or isoform thereof may be prepared by techniques known to those skilled in the art, including but not limited to, solvent evaporation and spray drying techniques. In some embodiments the microspheres are formed with a water-polymer ratio of between about 0.1 to about 1.0, such as but not limited to about 0.5 to about 1.0, about 0.55 to about 1.0, about 0.6 to about 1.0, about 0.65 to about 1.0, about 0.7 to about 1.0, about 0.75 to about 1.0, about 0.8 to about 1.0, about 0.85 to about 1.0, about 0.9 to about 1.0, or about 0.95 to about 1.0. In some embodiments, the water-polymer ratio of the microspheres is about 0.5, about 0.55, about 0.6, about 0.65, about 0.7, about 0.75, about 0.8, about 0.85, about 0.9, about 0.95, or about 1.0. These water polymer ratios may be adjusted to achieve a particular release profile based on the concentration of hCG or derivative or isoform thereof and/or the polymer used to form the microsphere. See Bos et al., Pharmaceutical Technology, October 2011:110-120.

In some embodiments, the microspheres have a diameter of at least about 1 μm, at least about 2 μm, at least about 5 μm, at least about 10 μm, at least about 20 μm, at least about 30 μm, at least about 40 μm, at least about 50 μm, at most about 50 μm, at most about 60 μm, at most about 70 μm, at most about 80 μm, at most about 90 μm, or at most about 100 μm. In some embodiments, the microspheres have a diameter between about 20 μm and about 100 μm, between about 30 μm and about 100 μm, between about 30 μm and about 50 μm, or between about 50 μm and about 100 μm.

In some embodiments, the concentration of hCG use for the preparation of microspheres is at least about 5 mg/ml, at least about 10 mg/ml, at least about 15 mg/ml, at least about 20 mg/ml, at least about 25 mg/ml, at least about 30 mg/ml, at least about 35 mg/ml, at least about 40 mg/ml, at least about 45 mg/ml, at least about 50 mg/ml, at least about 55 mg/ml, at least about 60 mg/ml, at least about 65 mg/ml, at least about 70 mg/ml, at least about 75 mg/ml, at least about 80 mg/ml, at least about 85 mg/ml, at least about 90 mg/ml, at least about 95 mg/ml, at least at least at least about 100 mg/ml. The concentration of hCG use for the preparation of microspheres may be about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, or more of protein.

### B1. hCG Microspheres Composed of PolyActive™ Multi-Block Copolymers

In certain aspects, the copolymer is a block copolymer and may, optionally, comprise or alternatively consist essentially of, polyethylene glycol (PEG) and one or more other polymers. In some aspects, the one or more other polymers may optionally be selected from polyethylene terephthalate (PET), polypropylene terephthalate, and polybutylene terephthalate (PBT). Non-limiting exemplary polymers are PolyActive™ polymers produced by OctoPlus and/or Dr. Reddy's Laboratories having a chemical structure of repeating polymer units, e.g.:

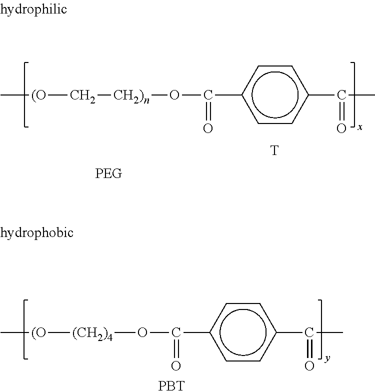

PolyActive™ polymers are represented by a series of poly(ether ester) multi-block copolymers, based on poly(ethylene glycol), PEG, and poly(butylene terephthalate), PBT. A major advantage of this system is the ability to vary the amount and length of each of the two building blocks, creating a diverse family of customized polymers. Polymer matrix characteristics such as rate of controlled release, degradation, swelling and strength can be precisely controlled by the appropriate combination of the two copolymer segments.

While PolyActive™ polymers have been used prior to the present invention in drug delivery systems, prior to the present invention the polymers had not been successfully formulated for a highly complex glycoprotein such as hCG. In particular, prior publications regarding PolyActive™ polymers state that the polymers are useful for the controlled release of “biopharmaceuticals and small lipophilic molecules.” P. Mansell, “OctoPlus broadens rights to PolyActive delivery technology,” In-Pharma Technologist.com, dated Apr. 26, 2007 (See http://www.in-pharmatechnologist.com/Ingredients/OctoPlus-broadens-rights-to-PolyActive-delivery-technology, downloaded on Mar. 9, 2016). Examples of biopharmaceuticals includes proteins.

When a PEG polymer is present in a hCG microsphere dosage form prepared of PolyActive PEG/PBT multi-block copolymers, the length of the PEG may be varied between from about 1000 to about 2500 g/mol. Non-limiting examples include PEG lengths of at least about 1000 g/mol, at least about 1100 g/mol, at least about 1200 g/mol, at least about 1300 g/mol, at least about 1400 g/mol, at least about 1500 g/mol, at least about 1600 g/mol, at least about 1700 g/mol, at least about 1800 g/mol, at least about 1900 g/mol, at most about 2000 g/mol, at most about 2100 g/mol, at most about 2200 g/mol, at most about 2300 g/mol, at most about 2400 g/mol, or at most about 2500 g/mol.

In some embodiments, PEG and the one or more other polymer present in the hCG microsphere dosage form prepared of PolyActive PEG/PBT multi-block copolymers are present in a ratio (by weight) between about 80/20 to about 60/40, such as but not limited to about 79/21 to about 60/40, about 78/22 to about 60/40, about 77/23 to about 60/40, about 76/24 to about 60/40, about 75/25 to about 60/40, about 74/26 to about 60/40, about 73/27 to about 60/40, about 72/28 to about 60/40, about 71/29 to about 60/40, about 70/30 to about 60/40, about 69/31 to about 60/40, about 68/32 to about 60/40, and about 67/33 to about 60/40. Examples of weight ratios include about 80/20, about 79/21, about 78/22, about 77/23, about 76/24, about 75/25, about 74/26, about 73/27, about 72/28, about 71/29, about 70/30, about 69/31, about 68/32, or about 67/33.

### B2. hCG Microspheres Composed of Multi-Block Copolymers Containing Crystalline Poly(L-Lactide) Building Blocks

Other exemplary polymers that can be utilized in the sustained release hCG compositions of the invention include SynBiosys® polymers produced by Innocore Pharmaceuticals. InnoCore's SynBiosys® technology offers a novel platform of bioresorbable polymers that are specifically designed to function as drug delivery systems. The polymers are composed of D,L-lactide, glycolide, ε-caprolactone and polyethylene glycol that have been approved for human applications. While SynBiosys® polymers have been used prior to the present invention in drug delivery systems, prior to the present invention the polymers had not been successfully formulated for a highly complex glycoprotein such as hCG.

SynBiosys® polymers are described in WO-A-2013/015685, the complete content of which is herewith incorporated by reference. The biodegradable, semi-crystalline, phase-separated, thermoplastic multi-block copolymers described therein comprise at least one hydrolysable pre-polymer (A) segment and at least one hydrolysable pre-polymer (B) segment, wherein said multi-block copolymer has a T_{g} of 37° C. or less and a Tₘ of 110-250° C. under physiological conditions, wherein the segments are linked by a multifunctional chain extender, wherein the segments are randomly distributed over the polymer chain, and wherein pre-polymer (A) segment comprises polyethylene glycol.

The morphology and properties under physiological conditions (i.e. in the body) may be different from the morphology and properties under ambient conditions (dry, room temperature). The transition temperatures, T_{g} and Tₘ, as used herein, refer to the corresponding values of a material when applied in vivo; viz. when at equilibrium with an atmosphere that is saturated with water vapor and at body temperature. This may be simulated in vitro by performing DSC measurement after allowing the material to equilibrate with a water-saturated atmosphere.

The pre-polymer (A) segment can comprise reaction products of ester forming monomers selected from diols, dicarboxylic acids, and hydroxycarboxylic acids. Preferably, the pre-polymer (A) segment comprises reaction products of glycolide, lactide (D and/or L), ε-caprolactone, and/or δ-valerolactone.

The pre-polymer (A) segment can have a Mₙ of about 500 g/mol or more, such as about 700 g/mol or more, about 1000 g/mol or more, about 2000 g/mol or more, about 3000 g/mol or more, or about 4000 g/mol or more. Typically, the pre-polymer (A) segment has a Mₙ of about 80 000 g/mol or less.

The pre-polymer (B) segment preferably comprises poly(L-lactide), more preferably poly(L-lactide) with a Mₙ of about 1000 g/mol or more, such as about 2000 g/mol or more, about 3000 g/mol or more, or about 4000 g/mol or more. Typically, the pre-polymer (B) segment has a Mₙ of about 80 000 g/mol or less.

The content of pre-polymer (A) in the multi-block copolymer can be from about 10% to about 90% based on total weight of the multi-block copolymer, such as from about 30% to about 75%, or from about 50% to about 70%.

The content of pre-polymer (B) in the multi-block copolymer can be from about 10% to about 90% based on total weight of the multi-block copolymer, such as from about 25% to about 70%, or from about 30% to about 50%.

The multifunctional chain extender can be a difunctional aliphatic chain extender, preferably a diisocyanate, such as 1,4-butane diisocyanate or 1,6-hexane diisocyanate.

The polyethylene glycol in the poly(L-lactide) based SynBiosys® multi-block copolymer can have a Mₙ of about 150 to about 5000 g/mol, such as about 200 g/mol to about 1500 g/mol, about 600 to about 1000 g/mol, about 400 to about 3000 g/mol, about 600 to about 1500 g/mol, about 600 to about 5000 g/mol, or about 1000 to about 3000 g/mol.

Preferably, the biodegradable multi-block copolymer has a swelling ratio under physiological conditions of about 1 to about 4, preferably about 1 to about 2, more preferably about 1 to about 1.5.

In an embodiment, the biodegradable multi-block copolymer is a [poly(ε-caprolactone)-co-polyethylene glycol-co-poly(ε-caprolactone)]-b-[poly(L-lactide)] multi-block copolymer.

### B2. hCG Microspheres Composed of Multi-Block Copolymers Containing Crystalline Poly(p-Dioxanone) Building Blocks

In certain aspects, the biodegradable multi-block copolymer comprises a biodegradable, phase separated, thermoplastic multi-block copolymer comprising at least one amorphous hydrolysable pre-polymer (A) segment and at least one semi-crystalline hydrolysable pre-polymer (B) segment, wherein
said multi-block copolymer under physiological conditions has a T_{g} of about 37° C. or less and a Tₘ of about 50 to about 110° C.;
the segments are linked by a multifunctional chain extender;
the segments are randomly distributed over the polymer chain; and
the pre-polymer (B) segment comprises a X-Y-X tri-block copolymer wherein Y is a polymerisation initiator, and X is a poly(p-dioxanone) segment with a block length expressed in p-dioxanone monomer units of about 7 or more.

X is preferably a poly(p-dioxanone) segment with a block length expressed in p-dioxanone monomer units of about 7 to about 35, such as about 8 to about 30, about 9 to about 25, about 10 to about 20, or about 12 to about 15.

At least part of the pre-polymer (A) segment can be derived from a water-soluble polymer, such as about 30% or more by total weight of pre-polymer (A), about 40 to about 95%, about 50 to about 90%, or about 60 to about 85%.

Pre-polymer (A) can, for instance, comprise a reaction product of cyclic monomers and/or non-cyclic monomers. Suitable non-cyclic monomers may, for example, be selected from the group consisting of succinic acid, gluratic acid, adipic acid, sebacic acid, lactic acid, glycolic acid, hydroxybutyric acid, ethylene glycol, diethylene glycol, 1,4-butanediol and/or 1,6-hexanediol. Suitable cyclic monomers may, for example, be selected from the group consisting of glycolide, lactide, ε-caprolactone, 5-valerolactone, trimethylene carbonate, tetramethylene carbonate, 1,5-dioxepane-2-one, 1,4-dioxane-2-one (p-dioxanone) and/or cyclic anhydrides, such as oxepane-2,7-dione.

Preferably, the pre-polymer (B) segment comprises a relatively large poly(p-dioxanone) part. For example, about 70% or more by total weight of the pre-polymer (B) segment may be poly(p-dioxanone), preferably about 80% or more, more preferably about 90% or more.

The pre-polymer (B) segment may have a number average molecular weight Mₙ of about 1300 to about 7200 g/mol, preferably about 1300 to about 5000 g/mol, more preferably about 1500 to about 4500 g/mol, even more preferably about 2000 to about 4000 g/mol, such as about 2200 to about 3000 g/mol.

The pre-polymer (B) segment may have a weight average molecular weight M_{w} of about 1800 to about 10800 g/mol, preferably about 1800 to about 7000 g/mol, more preferably about 2100 to about 6300 g/mol, even more preferably about 2600 to about 5600 g/mol, such as about 3000 to about 4200 g/mol.

The pre-polymer (B) segment may have a T_{g} of less than about 0° C., such as less than about −20° C., or less than about −40° C. The pre-polymer (B) segment may have a Tₘ in the range of about 60 to about 100° C., preferably in the range of about 75 to about 95° C.

The water-soluble polymer can be selected or derived from the group of polymers consisting of polyethers such as polyethylene glycol (PEG), polytetramethylene oxide (PTMO), polypropylene glycol (PPG), polyvinyl alcohol (PVA), polyvinyl pyrrolidone (PVP), polyvinyl caprolactam, poly(hydroxyethyl methacrylate) (poly-HEMA)), polyphosphazenes, or copolymers of these polymers. Preferably, the water-soluble polymer is derived from polyethylene glycol. More preferably, the water-soluble polymer is derived from polyethylene glycol having a Mₙ of about 150 to about 5000 g/mol.

The chain-extender may be a difunctional aliphatic chain-extender. Preferably, the chain-extender is a diisocyanate, such as 1,4-butane diisocyanate or hexamethylene diisocyanate.

In an embodiment, the biodegradable multi-block copolymer is a [poly(ε-caprolactone)-co-polyethylene glycol-co-poly(ε-caprolactone)]-b-[poly(p-dioxanone)] multi-block copolymer.

In certain aspects, the biodegradable multi-block copolymer is represented by [(R¹R²ₙR³)_{q}]ᵣ[(R⁴ₚR⁵R⁶ₚ)]ₛ,

wherein
R¹, and R³ are each

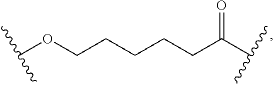 [SMILES: *OCCCCCC(*)=O]

### R² is

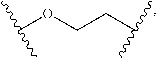 [SMILES: *CCO*]

R⁴ and R⁶ are each

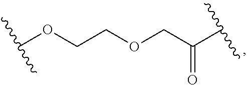 [SMILES: *OCCOCC(*)=O]

n, being the number of repeating R² moieties, is about 20 to about 115, preferably about 35 to about 100, more preferably about 45 to about 85;
p, being the number of repeating R⁴ and R⁶ moieties is about 7 or more, preferably about 7 to about 35, more preferably about 10 to about 20, even more preferably about 10 to about 14;
q, being the number average molecular weight of the (R¹R²ₙR³) block is about 1000 to about 7000 g/mol, preferably about 3000 to about 5000 g/mol, more preferably about 3800 to about 4200 g/mol;
r/s, being the ratio of pre-polymer (A) segment over pre-polymer (B) segment is about 0.10 to about 1.0, such as about 0.15 to about 0.50, or about 0.20 to about 0.30.

In an embodiment, the biodegradable multi-block copolymer is represented by [(R¹R²ₙR³)_{q}]ᵣ[(R⁴ₚR⁵R⁶ₚ)]ₛ, wherein each of R¹, R², R³, R⁴, R⁵, and R⁶ independently are as defined above, and wherein n is about 65 to about 71, p is about 11 to about 13, q is about 3800 to about 4200, r is about 15 to about 25, and, s is about 75 to about 85.

When a PEG polymer is present in the hCG microsphere dosage form prepared of a poly(p-dioxanone) based multi-block copolymers, the length of the PEG may be varied from between about 1000 to about 5000 g/mol. Non-limiting examples include PEG lengths of at least about 1000 g/mol, at least about 1200 g/mol, at least about 1400 g/mol, at least about 1600 g/mol, at least about 1800 g/mol, at least about 2000 g/mol, at least about 2200 g/mol, at least about 2400 g/mol, at least about 2600 g/mol, at least about 2800 g/mol, at least about 3000 g/mol, at least about 3200 g/mol, at least about 3400 g/mol, at least about 3600 g/mol, at least about 3800 g/mol, at most about 4000 g/mol, at most about 4200 g/mol, at most about 4400 g/mol, at most about 4600 g/mol, at most about 4800 g/mol, or at most about 5000 g/mol.

### C. Formulations

Aspects of the disclosure relate to formulations comprising a plurality of hCG microspheres. Such formulations may be comprised of a homogeneous or heterogeneous mixture of microspheres according to any one of the parameters disclosed herein. Further such formulations may optionally further comprise a pharmaceutically acceptable excipient and/or other components related to the specific indication being treated.

### D. Methods of Administration and Release Profile

A feature of the microspheres disclosed herein is a release profile that allows for extended release of hCG or a derivative or isoform thereof. In some aspects, less than or about 1/7 of the hCG or a derivative or isoform thereof in the microsphere or microsphere formulation is released in the first 24 hours post administration, such as less than or about 1/14, less than or about 1/21, less than or about 1/28, less than or about 1/29, less than or about 1/30, less than or about 1/31, less than or about 1/33, less than or about 1/34, less than or about 1/35, less than or about 1/42, less than or about 1/49, less than or about 1/56, less than or about 1/57, less than or about 1/58, less than or about 1/59, less than or about 1/60, less than or about 1/61, or less than or about 1/62. In some aspects, between about 2% to about 40% of the hCG or a derivative or isoform thereof in the microsphere or microsphere formulation is released in the first 24 hours, for example about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, about 15%, about 15.5%, about 16%, about 16.5%, about 17%, about 17.5%, about 18%, about 18.5%, about 19%, about 19.5%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, or any range comprising two of these values, e.g., about 18.5% to about 26%.

Without wishing to be bound by any theory, it is envisioned that the release profiles described above allow for extended release of hCG or a derivative or isoform thereof for specified periods, such as, but not limited to, about one week or more, about two weeks or more, about three weeks or more, about four weeks or more, about five weeks or more, about six weeks or more, about seven weeks or more, about eight weeks or more, about one month or more, about two months or more, or about 7 days or more, about 8 days or more, about 9 days or more, about 10 days or more, about 11 days or more, about 12 days or more, about 13 days or more, about 14 days or more, about 15 days or more, about 16 days or more, about 17 days or more, about 18 days or more, about 19 days or more, about 20 days or more, about 21 days or more, about 22 days or more, about 23 days or more, about 24 days or more, about 25 days or more, about 26 days or more, about 27 days or more, about 28 days or more, about 29 days or more, about 30 days or more, about 31 days or more, about 32 days or more, about 33 days or more, about 34 days or more, about 35 days or more, about 36 days or more, about 37 days or more, about 38 days or more, about 39 days or more, about 40 days or more, about 41 days or more, about 42 days or more, about 43 days or more, about 44 days or more, about 45 days or more, about 46 days or more, about 47 days or more, about 48 days or more, about 49 days or more, about 50 days or more, about 51 days or more, about 52 days or more, about 53 days or more, about 54 days or more, about 55 days or more, about 56 days or more, about 57 days or more, about 58 days or more, about 59 days or more, about 60 days or more, about 61 days or more, or about 62 days or more, about 1 month or more, about 2 months or more, about 3 months or more, about 4 months or more, about 5 months or more, about 6 months or more.

It is envisioned that the microspheres and formulations comprising these microspheres may be administered according to any mode of administration known in the art, including but not limited to topical, enteral, parenteral, oral, sublingual, via inhalation, nasal, via injection, intradermal, transdermal, intramuscular, subcutaneous, bolus dose, infusion, and/or any other suitable method.

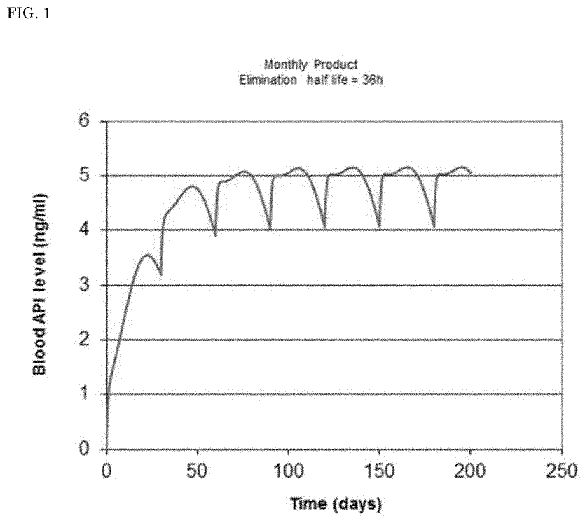

In certain aspects, the disclosure relates to methods of administration that achieve or approximate the theoretical release profile according to FIG. 1. In some embodiments, the release profile exhibits minimal or no burst release.

Aspects of the disclosure relate to regimens of administration that approximate a release profile, for example according to FIG. 1. Such regimens include administration of the extended release hCG formulation about every one week, two weeks, three weeks, four weeks, five weeks, six weeks, seven weeks, eight weeks, one month, two months, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, or about 62 days.

### E. Methods of Treatment

The hCG microspheres and formulations thereof disclosed herein may be used to treat a variety of treatments and administered according to appropriate periodicity and dose based on the indication. Indications may be human or veterinary. Exemplary indications include hyogonadotropism, cryptorchidism, luteal phase maintenance (e.g. in assisted reproduction techniques), contraception, weight loss, pituitary gland disorders, breast cancer and/or any other disease or disorder associated with hCG deficiency.

Certain embodiments relate to the treatment of breast cancer with the hCG microspheres and formulations disclosed herein. Pregnancy is known to be protective against breast cancer. Not to be bound by theory, it is expected that administering the hCG microspheres and formulations disclosed herein could achieve up to an about 30% to about 40% reduction of breast cancer risk. See Russo and Russo, Molecular Basis of Breast Cancer: Prevention and Treatment (Springer Science & Business Media, 2004). Further, unlike methods disclosed in the art for administration of hCG, the hCG microspheres and formulations disclosed herein require fewer administrations, e.g. less than about 10, less than about 9, less than about 8, less than about 7, less than about 6, less than about 5, less than about 4, less or than about 3 administrations, or about 1 or 2 administrations, versus 45 administrations of conventional hCG over 3 months. In other embodiments, the described formulations can be effective with a 1×per week administration, or 1×, 2× or 3× per month administration schedule for treating and/or preventing breast cancer. In some embodiments, the treatment is for existing breast cancer in nulliparous women. In some embodiments, the described formulations are in the form of one injection providing a therapeutic effect longer than one month

### F. General Definitions

As used in the description of the invention and the appended claims, the singular forms “a,” “an” and “the” are intended to include the plural forms as well, unless the context clearly indicates otherwise. The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

As used herein, the term “comprising” is intended to mean that the formulations and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising”. “Consisting of” shall mean excluding more than trace elements of other ingredients and substantial method steps for administering the formulations disclosed herein. Aspects defined by each of these transition terms are within the scope of the present disclosure.

A “formulation” is intended to mean a combination of active agent and another compound or composition, inert (for example, a detectable agent or label) or active, such as an adjuvant.

A “pharmaceutical formulation” is intended to include the combination of an active agent with a carrier, inert or active, making the formulation suitable for diagnostic or therapeutic use in vitro, in vivo or ex vivo.

“Pharmaceutically acceptable carriers” refers to any diluents, excipients, or carriers that may be used in the formulations of the invention. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The term “extended release” is used herein to refer to the ability to release an active ingredient (hCG) over a specified period of time. The term “burst release” refers to a phase of rapid release of the active ingredient (hCG) into the environment, such that continued release over an extended period of time is not sustainable.

As used herein, the terms “subject” or “patient” are used interchangeably to mean any animal. In some embodiments, the subject may be a mammal; in further embodiments, the subject may be a human, mouse, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. For instance, descriptors may be used to refer to biological material (e.g. tissue, organoids, samples) exhibiting characteristics of a particular organ, e.g. the use of “hepatic” to describe liver-derived tissue or a liver-like organoid. While not explicitly defined by below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### V. EXAMPLES

The following examples are non-limiting and illustrative of procedures which can be used in various instances in carrying the disclosure into effect. Additionally, all references disclosed herein are incorporated by reference in their entirety.

### Example 1—Comparison of Commercially Available hCG

Ovidrel® obtained from EMD Serono (“Serono”) and Dong-A hCG were compared for differences in molecular weight.

Colloidal Blue native and SDS page gel were prepared. Gels suitable for Silverstaining were also prepared but generated unreadable results.

Under native gel conditions with native sample and running buffer, the prepared samples of the API hCG of both Dong-A and Serono showed the same band pattern.

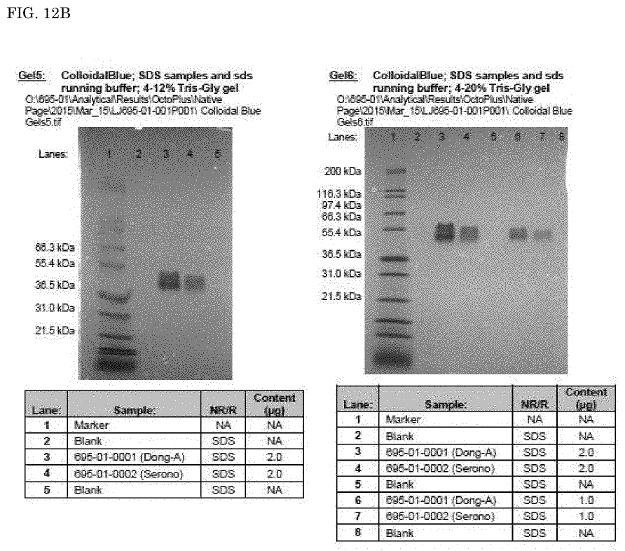

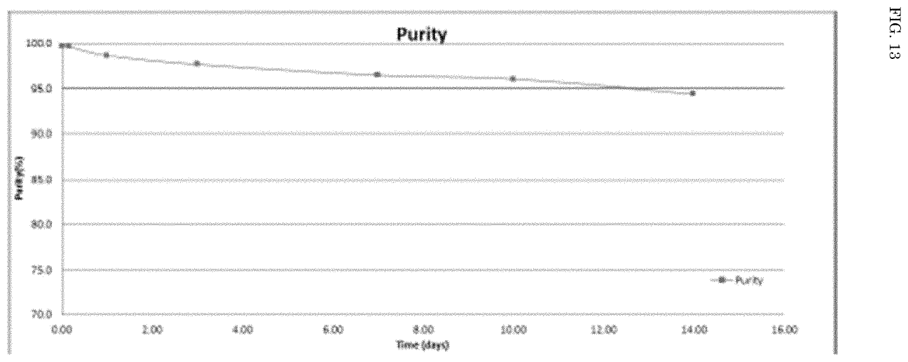

Native PAGE analysis demonstrated that there are no significant molecular weight differences between hCG from Dong-A and Serono. The molecular weight on Native PAGE gel is similar, but difficult to read due to a non-compatible marker. In non-native and semi-native conditions (presence of SDS), molecular weight of hCG from Dong-A and Serono (banding between 44 and 52 kDa) are similar to the listed single diffuse band of 50-60 kDa (FIG. 12).

### Example 2—Production of PolyActive hCG Microspheres

Dong-A hCg was concentrated to a target concentration of 20 mg/ml using PBS buffer+methionine at pH 7.4. A summary of the analytics is presented in Table 1 below.

TABLE 1
hCG Concentration Analysis

| Analysis | Parameters | Results |
|---|---|---|
| Visual | Insoluble Particles | No visible particles |
| PSA | Z-avg | 8.66 nm |
| | Pdl | 0.059 |
| HP-SEC | Integrity | No aggregates peak |
| | Degradation | No detectable |
| | Concentration | 21.5 mg/ml |

The integrity of the protein was maintained during the concentration step, resulting in about 20 mg/ml (21.5 mg/ml) protein solution which in turn allows for a protein/polymer ratio of 1.5-2%. The final drug product should thus be in the range of between about 0.2-0.6 mg hCG in 0.2 cc. The integrity of the protein was maintained during concentration (FIG. 11).

The concentrated hCG was introduced into a primary emulsion with an organic solution comprising the polymer (PolyActive™ polymer (1 g/9 g dichloromethane)) using a positive displacement pipet and then homogenized at 19 000 rpm for 30 seconds at room temperature. This primary emulsion was then mixed with PBS PVA 7% for 5 min and then PBS+methionine was added for 5 hours followed by 5 washings. A similar process was repeated with placebo (PBS+methionine) loaded microspheres.

Microspheres are generated according to this method for a variety of polymers, including those listed below:

PolyActive™ PEG 1500 series: PEG/PBT weight ratio=70/30, 75/25, 80/20, 90/10
PolyActive™ PEG 1000 series: PEG/PBT weight ratio=70/30, 67/33
PolyActive™ PEG 2000 series: PEG/PBT weight ratio=60/40, 75/25, 80/20

The resulting particles were sized using a Mastersizer as described in Table 2.

TABLE 2
Microsphere Size Distribution
Placebo

| | Size | | |
|---|---|---|---|
| PolyActive | Distribution (μm) | | |
| PEG/PBT ratio | D_{0.1} | D_{0.5} | D_{0.9} |
| 2000/67/33 | 4.88 | 22.09 | 45.85 |
| 2000/75/25 | 50.60 | 98.60 | 174.30 |
| 2000/80/20 | 37.36 | 64.53 | 103.91 |
| 1500/70/30 | 25.04 | 48.50 | 86.81 |
| 1500/75/25 | 48.80 | 83.34 | 144.12 |
| 1500/80/20 | 43.90 | 74.35 | 123.71 |
| 1500/90/10 | 41.44 | 141.67 | 271.80 |
| 1000/67/33 | 27.63 | 58.65 | 104.93 |
| 1000/70/30 | 27.85 | 59.34 | 106.37 |
| 1500/70/30 | 35.39 | 69.60 | 119.90 |
| 1500/75/25 | 37.32 | 78.78 | 139.33 |
| 1500/80/20 | 24.60 | 61.53 | 112.19 |
| 1000/67/33 | 23.06 | 51.14 | 98.40 |
| 1000/70/30 | 34.10 | 67.05 | 116.1 |

Microscopy confirmed the results obtained with the Mastersizer. No significant morphological differences were observed, except for the microsphere formulation with the weight ratio 1500/90/10 that gave large ovoid microsphere shapes. Based on size distribution, the formulations of Table 3, in general those that ranged in size between about 50 and 100 μm without any extra processing, were selected for further analysis.

TABLE 3
Microspheres for Further Analysis

| | | | PA theoretical | rhCG theoretical |
|---|---|---|---|---|
| Formulation | Batch No | PA ratio | final concentration | final concentration |
| PA Placebo | F695-01-003p009B | 1500/80/20 | 15 mg/ml | NA |
| PA Placebo | F695-01-003p009C | 1500/75/25 | 15 mg/ml | NA |
| PA Placebo | F695-01-003p011B | 1000/67/33 | 15 mg/ml | NA |
| PA Placebo | F695-01- | 1500/70/30 | 15 mg/ml | NA |
| | 003p009G | | | |
| Encapsulated rhCG | F695-01-003p010B | 1000/67/33 | 15 mg/ml | 0.4 mg/ml |
| Encapsulated rhCG | F695-01-003p010C | 1500/80/20 | 15 mg/ml | 0.4 mg/ml |
| Encapsulated rhCG | F695-01-003p009E | 1500/70/30 | 15 mg/ml | 0.4 mg/ml |
| Encapsulated rhCG | F695-01-003p009F | 1500/75/25 | 15 mg/ml | 0.4 mg/ml |

### Example 3—Analysis of Release Profiles of PolyActive hCG Microspheres

Microspheres were prepared according to the method of Example 2. A series of formulations listed in Table 4 were tested via an in vitro release assay.

TABLE 4
Round 1 of in vitro release
Sample ID's/Description

| | Formulation/Batch | | | Target hCG | Total volume |
|---|---|---|---|---|---|
| Sample ID | number | Description | Vials | (μg/ml) | in vial (ml) |
| 695-01-0006 | F695-01-003P010B | Formulation 1 | 2 | 400 | 2.0 |
| | | Active Batch PA 1000/67/33 | | | |
| 695-01-0009 | F695-01-003P010C | Formulation 1 | 2 | 400 | 2.0 |
| | | Active Batch PA 1500/80/20 | | | |
| 695-01-0010 | F695-01-003P010E | Formulation 1 | 2 | 400 | 2.0 |
| | | Active Batch PA 1500/70/20 | | | |
| 695-01-0011 | F695-01-003P010F | Formulation 1 | 2 | 400 | 2.0 |
| | | Active Batch PA 1500/75/25 | | | |
| 695-01-0012 | DA-3803B153 | Control sample: | 1 | ~50 | 0.5 |
| | | ~50 μg/ml hCG in IVR | | | |
| | | medium | | | |

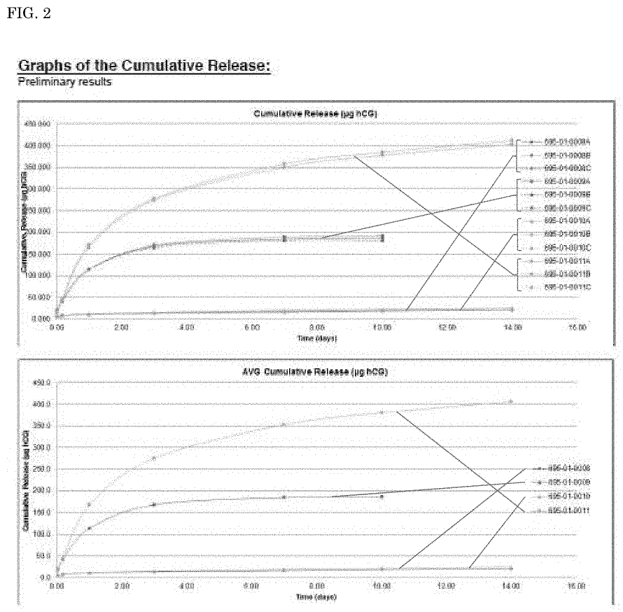

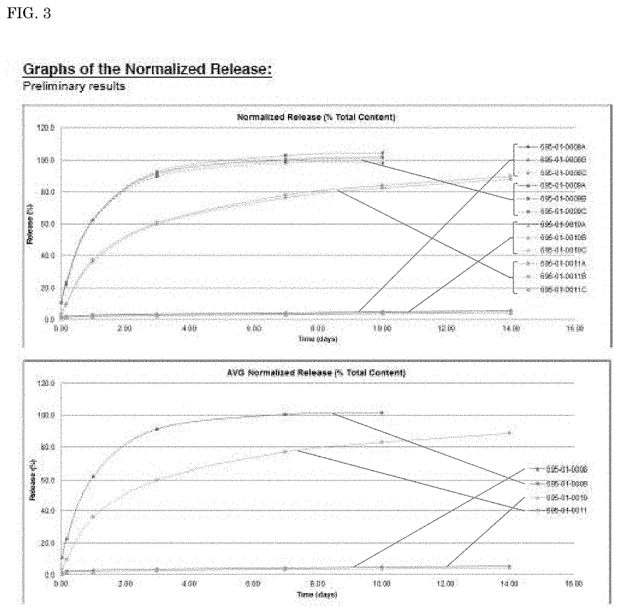

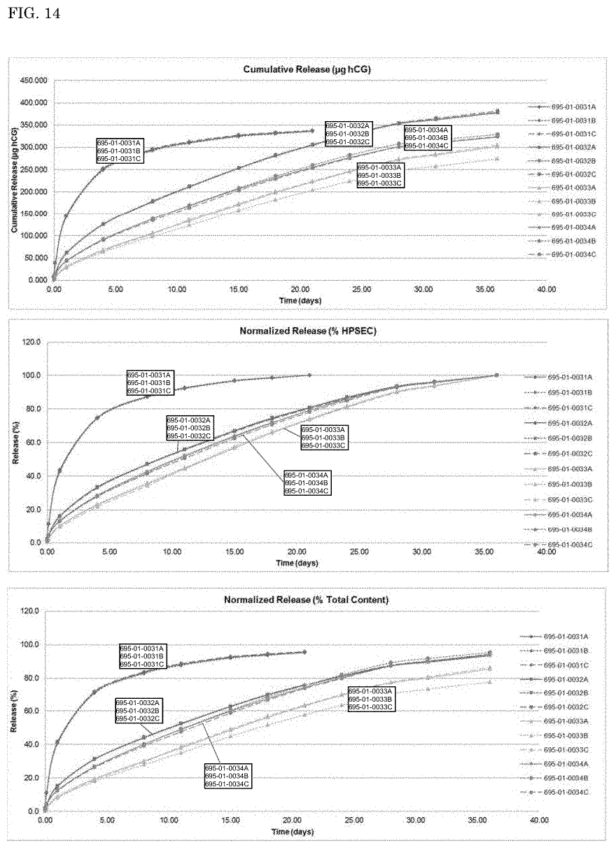

Samples and a control were incubated at 37° C. with 0.05% NaN₃ and 0.05% Tween20 in PBS at pH 7.4. Preliminary cumulative and normalized release profiles were determined, carried out to 14 days (FIGS. 2-3). Formulations 695-01-0008, 695-01-0010, and 695-01-0011 all demonstrated low to no burst release. The hCG remained substantially pure for the control formulation (FIG. 13), indicated by Table 5, decreasing only about 5% over 14 days.

TABLE 5
Purity of hCG

695-01-0012

| | | Content by | Purity by | Recovery |
|---|---|---|---|---|
| Time | | HPSEC | HPSEC | to |
| Time Point | days | (μg/ml hCG) | (% hCG) | TP0 (%) |
| 0 | 0.00 | 48.6 | 99.7 | 100 |
| 1 | 0.17 | 48.9 | 99.7 | 101 |
| 2 | 1.00 | 47.8 | 98.7 | 98 |
| 3 | 3.00 | 48.5 | 97.7 | 100 |
| 4 | 7.00 | 47.9 | 96.5 | 99 |
| 5 | 10.00 | 44.2 | 96.0 | 91 |
| 6 | 14.00 | 45.4 | 94.4 | 94 |

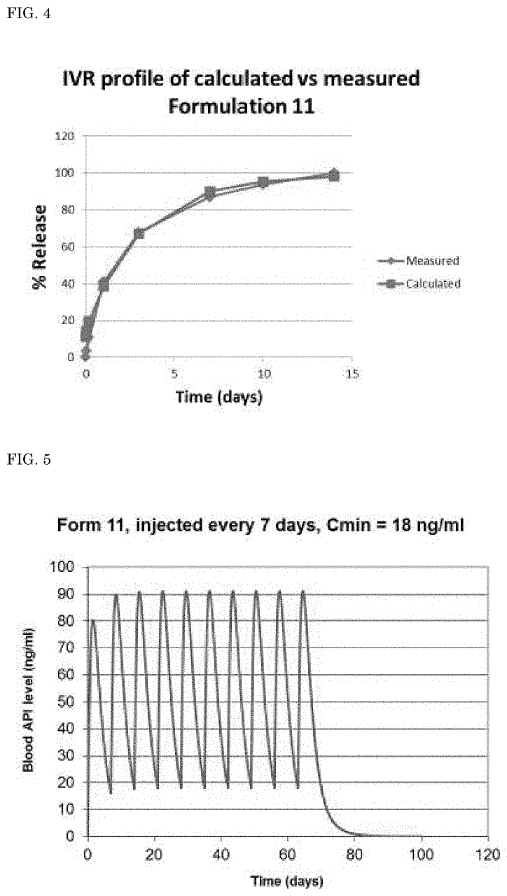

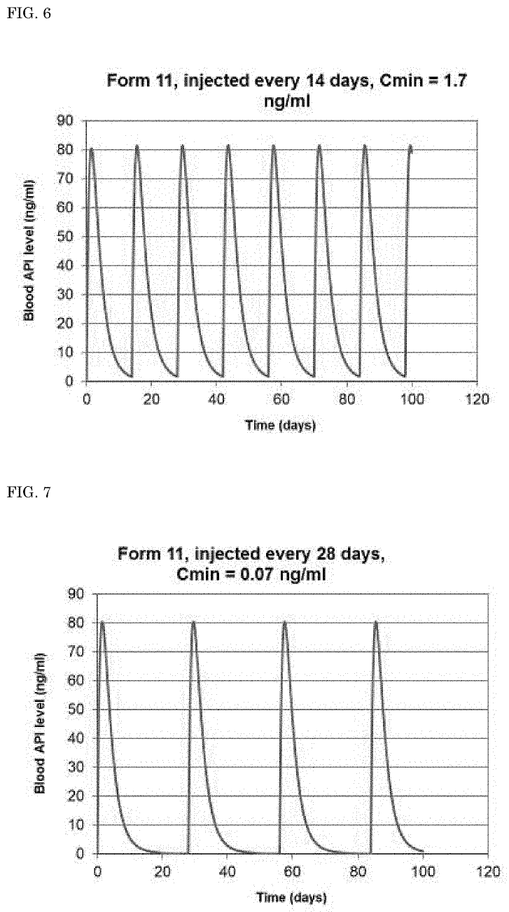

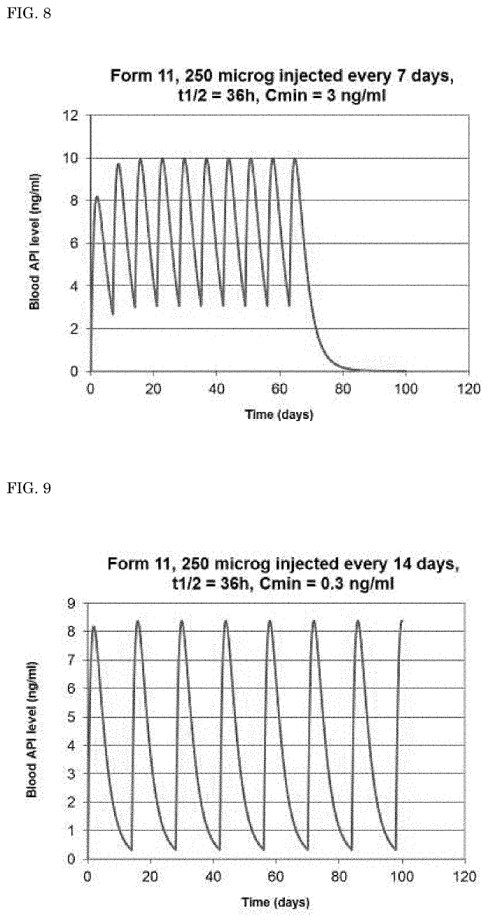

Formulation 695-01-0011 (“Formulation 11”) exhibited a suitable release profile for extended release hCG administration. Theoretical release profiles were determined for varying hCG doses of formulation 11 (FIGS. 5-9) based on its actual in vitro release profile (FIG. 4), assuming Bioavailability=0.5; Volume of distribution=5.5 l; Absorption half-life=8 h; Terminal elimination half-life=24 h; Max. microsphere density for injectability with 23-25 G needle is 15% w/v (150 mg/ml); 2% drug load; 1 cc injection delivers 3 mg hCG; and One injection: 3 mg of hCG in 1 ml formulated as “Formulation 11” PolyActive microspheres. Table 6 shows how half-life of hCG and dose interval result in minimum sustained hCG levels in blood in ng/ml for a product with 3 mg of hCG in Formulation 11.

TABLE 6
Estimated hCG levels in Blood
based on Formulation 11
Theoretical Release Profile
based on above assumptions

| | Dose interval (d) | | |
|---|---|---|---|
| t^{1/2} (h) | 7 | 14 | 28 |
| 24 | 18 | 1.7 | 0.07 |
| 30 | 27 | 2.7 | 0.1 |
| 36 | 37 | 4 | 0.14 |

The ratio between Cₘᵢₙ and Cₘₐₓ also shows large differences t^{1/2}=24 and 36 hrs. The plasma levels are proportional with the dose administered and the elimination half life. A formulation with a lower release rate, even up to 4 or 6 weeks, for a biweekly dosing could offer a lower Cₘₐₓ/Cₘᵢₙ ratio and more efficient use of the dose and would reduce the need for a higher loading % of hCG.

Thus, another round of in vitro release was considered, varying the parameters of functional formulation 11. The proposed variations are listed in Table 7. Further considerations include blending PolyActive™ PEG 1500 75/25 and PolyActive™ PEG 1500 70/30 at a weight ratio of 1:4, 1:1, 1.5:1, or 4:1 to mimic PolyActive™ PEG 1500 71/29, 72.5/27.5, 73/27, and 74/26, respectively.

TABLE 7
Proposed Variations of Formulation 11

| | Water/polymer | Particle | Target [hCG] for | | |
|---|---|---|---|---|---|
| Polymer | ratio | size | loading | Expected profile | Considerations |
| 1500- | 1.00 | 50-100 μm | Identical to | Identical to | Replicability |
| PEG75/PBT20 | | | Formulation 11 | Formulation 11 of | |
| | | | (target 20 mg/ml) | Round 1 | |
| 1500- | 0.75 | | | slower than | Lower water |
| PEG75/PBT25 | | | | Formulation 11 (2-4 | content may |
| | | | | wks?) | slow down |
| | | | | | release |
| 1500- | 0.50 | | | Much slower than | Lower water |
| PEG75/PBT25 | | | | Formulation 11 (4-6 | content may |
| | | | | wks?) | release |
| 1500- | 0.50 | 30-50 μm | | Slower than | Smaller particle |
| PEG75/PBT25 | | | | Formulatoin 11 (2-4 | sizze may speed |
| | | | | wks?) | up release |
| 1500- | 0.50 | 50-100 μm | | 2-4 wks release? | Polymer has |
| PEG77/PBT23 | | | | | prove safety |
| 1500- | 0.75 | | | 1-2 wks release? | Polymer has |
| PEG77/PBT23 | | | | | proven safety |

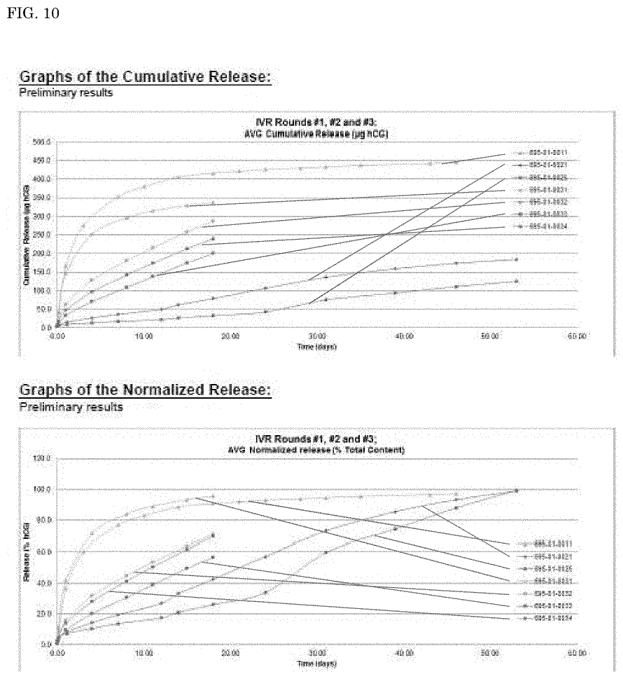

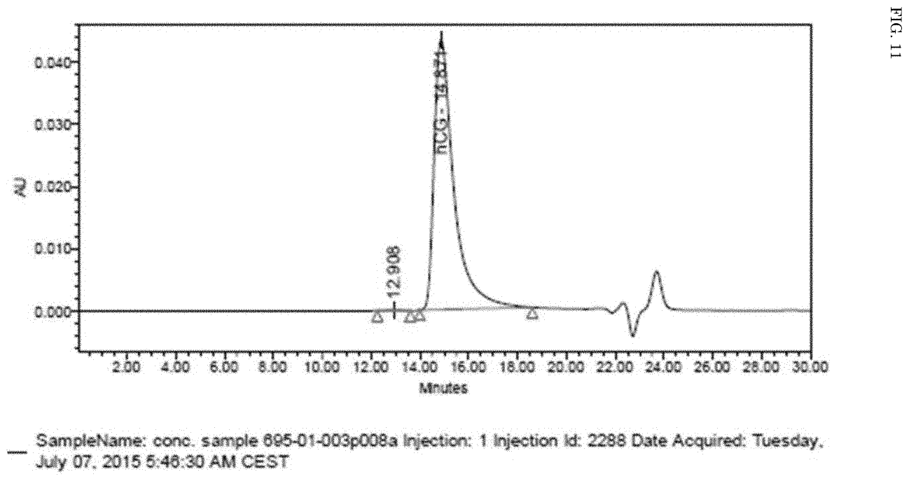

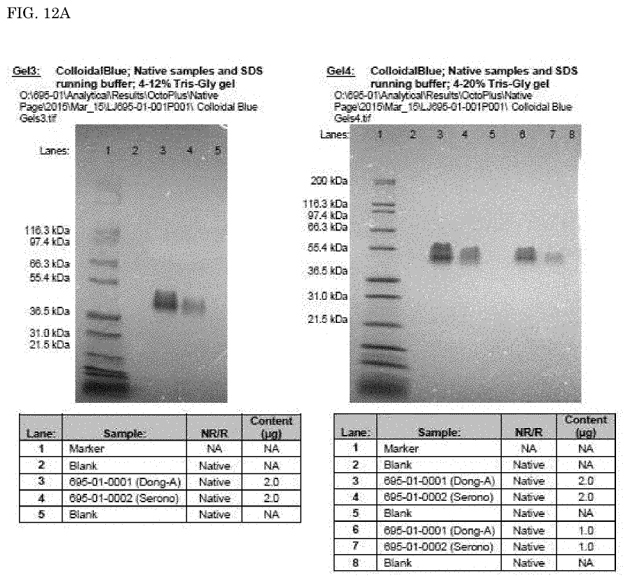

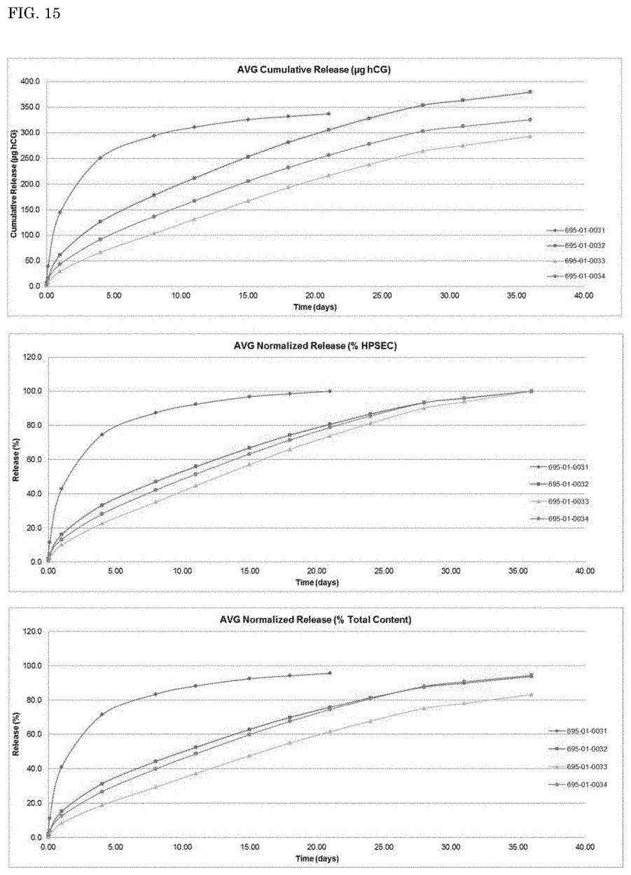

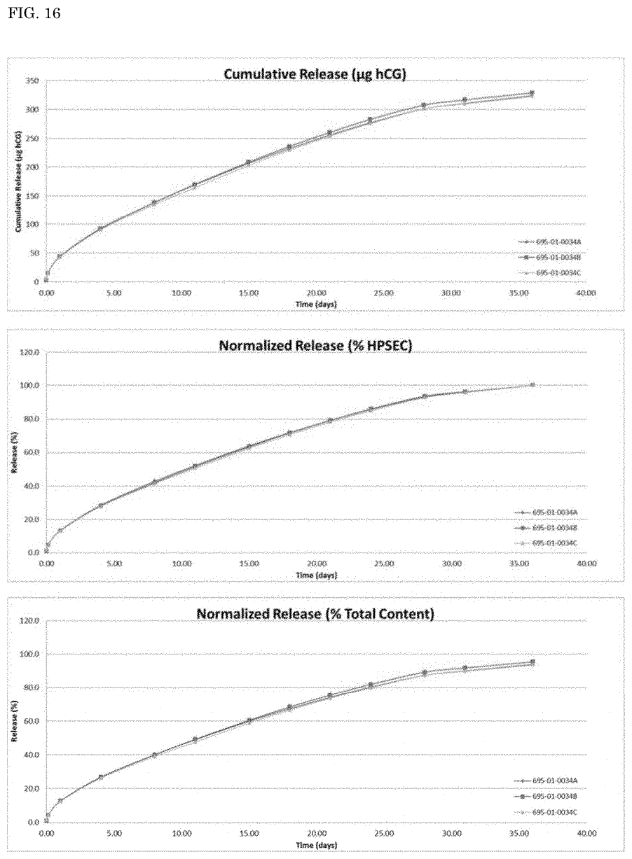

A second and third round of in vitro release assays were carried out for the additional samples listed in Table 8. A number of formulations—695-01-0021 (at least 4 to 6 week release), 695-01-0025, 695-01-0031, 695-01-0032, 695-01-0033 (possibly optimal), and 695-01-0034 exhibited hCG release profiles suitable for extended release durations ranging between 1 week to over 50 days. (FIGS. 10, 14-16). Further analysis of these formulations is provided in Table 8.

TABLE 8
Characterization of Extended Release Microspheres

| | | | | | | | | |
|---|---|---|---|---|---|---|---|---|
| Active Batch | 1500- | 1.0 | 458 | 4.2 % | IVR1 | 695-01-0011 | 38% release after 24 h | |
| PA1500/75/25 | PEG75/PBT25 | | | | | | | |
| Active Batch | 1500- | 1.0 | 226 | 1.74 % | IVR2 | 695-01-0020 | Different release profile; very low; | decide on |
| PA1500/75/25 | PEG75/PBT25 | | | | | | ~22% after 18 days; appears linear. IVR | continuation |
| | | | | | | | study was stopped after 18 days | beyond 31 days |
| Active Batch | 1500- | 1.0 | 363 | 3.76 % | IVR3 | 695-01-0031 | 42 % release after 24 h | |
| PA1500/75/25 | PEG75/PBT25 | | | | | | | |
| Loading 2.44% | | | | | | | | |
| Active Batch | 1500- | 0.75 | 186 | 1.43 % | IVR2 | 695-01-0021 | Most promising; ~80% after 31 days; | decide on |
| PA1500/75/25 | PEG75/PBT25 | | | | | | linear release. IVR was continued | continuation |
| W/P 0.75 | | | | | | | from 18 to 31 days | beyond 31 days |
| Active Batch | 1500- | 0.75 | 404 | 2.39 % | IVR3 | 695-01-0032 | | |
| PA1500/75/25 | PEG75/PBT25 | | | | | | | |
| Loading 2.0% | | | | | | | | |
| Active Batch | 1500- | 0.625 | 353 | 1.81 % | IVR3 | 695-01-0033 | 9% after 24 h | |
| PA1500/75/25 | PEG75/PBT25 | | | | | | | |
| Loading 1.67 % | | | | | | | | |
| Active Batch | 1500- | 0.5 | 126 | 1.005 % | IVR2 | 695-01-0025 | looked promising initially (linear) but lower | |
| PA1500/77/23 | PEG77/PBT23 | | | | | | release than 695-01-0021 and increases more | |
| | | | | | | | steeply towards the end (potentially related to | |
| | | | | | | | polyactive type; 60 % released after 31 days. | |
| | | | | | | | WR was continued from 18 to 31 days | |
| Active Batch | 1500- | 0.625 | 345 | 1.86 % | IVR3 | 695-01-0034 | 16% after 24 h | |
| PA1500/77/23 | PEG77/PBT23 | | | | | | | |
| Loading 1.67 % | | | | | | | | |

### Example 4—Production of hCG Extended Release Microspheres Composed of SynBiosys [PCL-Co-PEG-Co-PCL]-b-[PLLA] Multi-Block Copolymers

This example describes the preparation and characterization of hCG extended release microspheres prepared of SynBiosys [poly(ε-caprolactone)-co-polyethyleneglycol-co-poly(ε-caprolactone)-b-[poly(L-lactide)] (PCL-co-PEG-co-PCL]b-[PLLA]) multi-block copolymers.

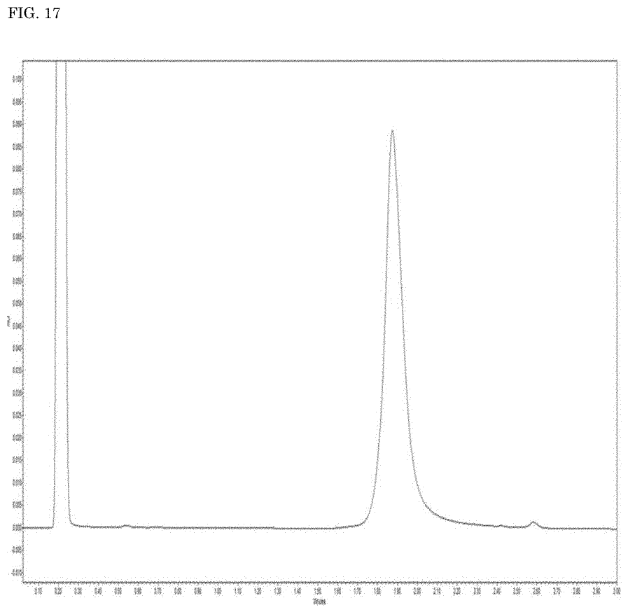

hCG (Dong-A Pharmaceutical Co., Ltd; Korea) was concentrated to a target concentration of 30 mg/ml using PBS buffer+methionine at pH 7.4 and Amicon Ultra −15 vials with a polyethersulfone membrane with a cut-off size of 10 kDa. The hCG concentration of the concentrated solution was 30.7 mg/ml hCG as determined with RP-UPLC. Visual examination of the concentrated protein solution showed that there were no insoluble particles. The integrity of hCG was maintained during the concentration step. RP-UPLC analysis (FIG. 17, UPLC chromatogram) confirmed the absence of aggregates and there were no signs of degradation.

Approximately 0.35 g of concentrated hCG solution was added to a solution of 0.50 g polymer in 2.92 g dichloromethane (DCM) and then homogenized at 22 000 rpm for 40 seconds to yield a water-in-oil (W/O) primary emulsion. This primary emulsion was then emulsified with a 4.0% aqueous PVA solution containing 5.0 w/v % NaCl using a continuous flow reactor thereby forming a water-in-oil-in-water (W/O/W) double emulsion. The W/O/W emulsion was stirred for 3 hours at room temperature to allow extraction and evaporation of dichloromethane. After completion of solvent evaporation, the hCG microspheres were collected by filtration and lyophilized to yield dry hCG microspheres.

Using the above described procedure, microspheres were prepared of blends of SynBiosys 50[PCL-co-PEG1000-co-PCL]2000-b-[PLLA]4000 and SynBiosys 30[PCL-co-PEG3000-co-PCL]4000-b-[PLLA]4000 at different formulation and process parameter settings (polymer blend ratio, polymer concentration, CP: DP ratio).

SynBiosys 50[PCL-co-PEG1000-co-PCL_]2000-b-[PLLA]4000 (also abbreviated as 50CP10C20-LL40) is a multi-block copolymer composed of a hydrophilic [PCL-co-PEG1000-co-PCL] prepolymer segment (A) with a molecular weight of 2000 g/mol (containing 50 mole % of polyethylene glycol with a molecular weight of 1000 g/mol) and a semi-crystalline poly(L-lactide) pre-polymer segment (B) with a molecular weight of 4000 g/mol which are chain-extended in a 50/50 wt. % block ratio by 1,4-butanediisocyanate. SynBiosys 30[PCL-co-PEG3000-co-PCI]4000-b-[PLLA]4000 (also abbreviated as 30CP30C40-LL40) is a multi-block copolymer composed of a hydrophilic [PCL-co-PEG3000-co-PCL] pre-polymer segment (A) with a molecular weight of 4000 g/mol (containing 75 mole % of polyethyleneglycol with a molecular weight of 3000 g/mol), and a semi-crystalline poly(L-lactide) pre-polymer segment (B) with a molecular weight of 4000 g/mole which are chain-extended in a 30/70 wt. % block ratio by 1,4-butanediisocyanate
The resulting hCG microspheres were characterized for their particle size distribution using a Coulter Counter Multisizer III. The average particle size varied from 21 to 48 μm (Table 9).

TABLE 9
Overview of hCG microspheres made of blends of
50CP10C20-LL40 and 30CP30C40-LL40.

| | | | | Size distribution (μm; CV %) | | hCG | EE |
|---|---|---|---|---|---|---|---|
| Batch nr | Pol ratio* | Pol. Conc. | CP:DP ratio | D50 (μm) | CV (%) | (wt. %) | (%) |
| JA16040 | 1:2 | 10% | 161 | 21 | 57 | 1.76 | 92 |
| JA16041 | 1:1 | 10% | 159 | 27 | 72 | 1.58 | 82 |
| JA16042 | 2:1 | 10% | 165 | 23 | 61 | 1.47 | 74 |
| JA16043 | 1:2 | 15% | 160 | 38 | 63 | 1.56 | 90 |
| JA16044 | 1:1 | 15% | 160 | 30 | 67 | 1.29 | 85 |
| JA16045 | 2:1 | 15% | 168 | 48 | 57 | 1.38 | 95 |
| JA16047 | 2:1 | 15% | 94 | 38 | 57 | 1.55 | 77 |
| JA16049 | 2:1 | 15% | 54 | 35 | 57 | 1.53 | 76 |

*Pol ratio = weight ratio Polymer 1/Polymer 2; polymer 1 = 50CP10C20-LL40; Polymer 2 = 30CP30C40-LL40

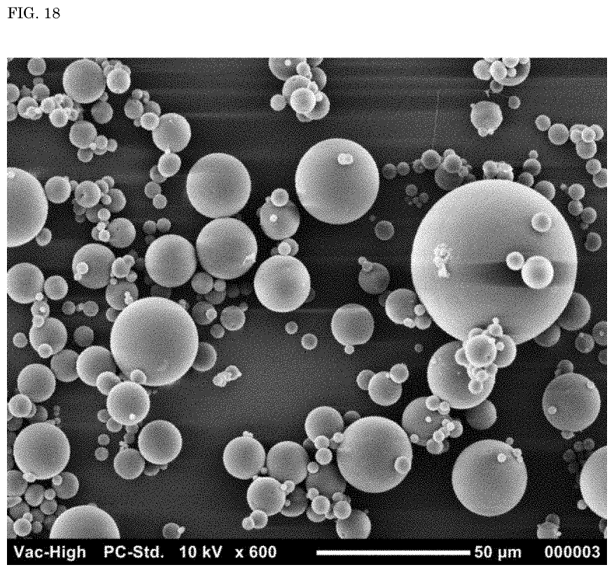

Microscopic examination of the hCG microspheres as evaluated by scanning electron microscopy using a JEOL JCM-5000 Neoscope confirmed the results with the Coulter Counter. All hCG microspheres had similar morphological characteristics, i.e. spherically shaped microparticles with a smooth surface (FIG. 18).

hCG content and encapsulation efficiency (EE) of all microspheres were determined by hydrolysis of the polymer in 0.1 M NaOH, mixing of the hydrolysate with 100 mM phosphate buffer pH 7.4 and subsequent analysis of the hCG concentration by RP-UPLC. The hCG content of the hCG microsphere batches varied between 1.3% and 1.8% representing encapsulation efficiencies of 75% to 95% (Table 9).

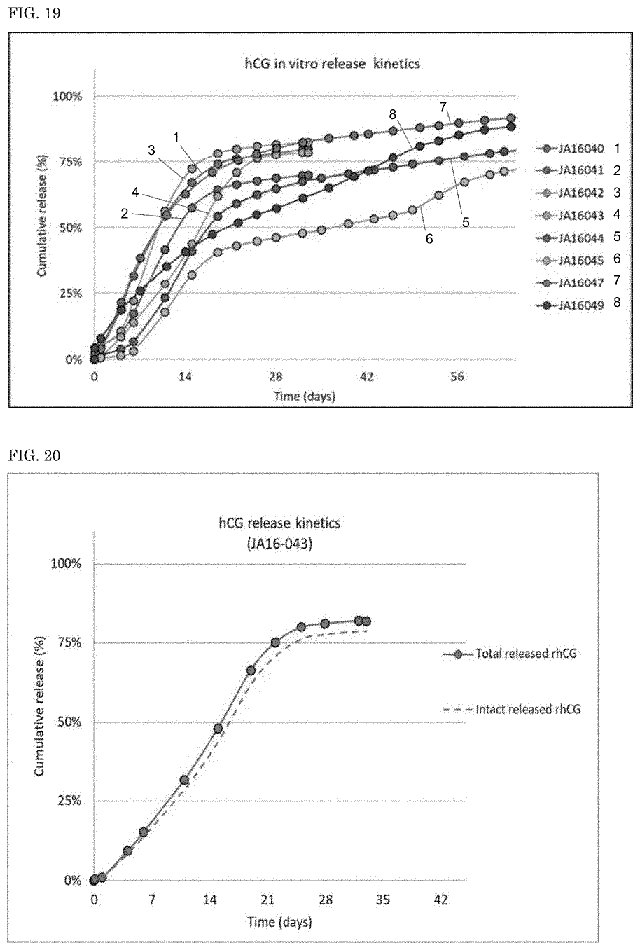

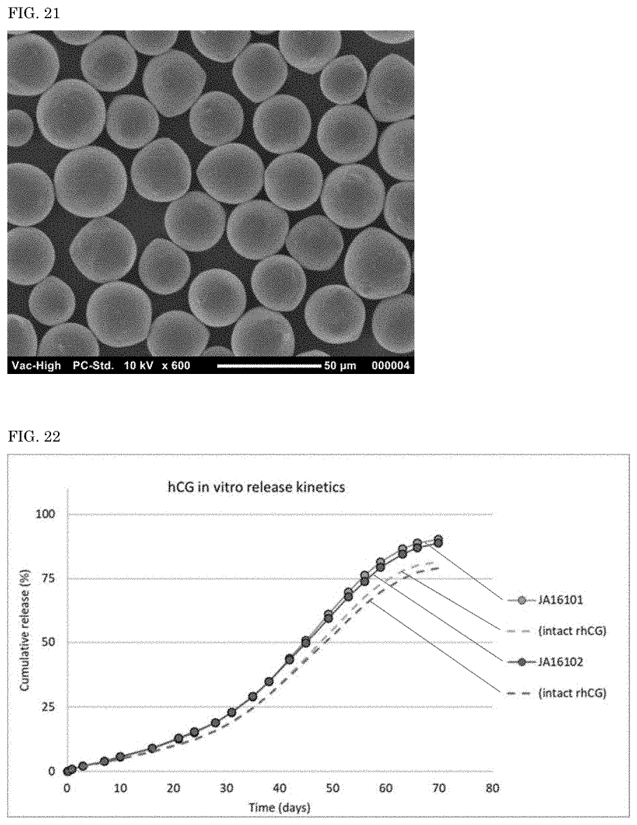

The in vitro release kinetics were determined by incubating hCG microspheres at 37° C. in PBS buffer pH 7.4 containing 0.01% Tween-20 and 0.01% sodium azide, sampling at predetermined and analysis of concentration hCG with RP-UPLC. Cumulative release profiles were determined, carried out to at least 4 weeks (FIG. 19). All formulations demonstrated low to no burst release, followed by gradual release of hCG thereafter. Some of the formulations showed a lag time followed by an irregular release pattern (JA16044 and JA16045). JA16043 exhibited the most promising release kinetics as it had no burst, linear release for a weeks and a recovery>75%. JA16-043 was further analyzed for the integrity of released hCG. The concentration of intact hCG was determined by RP-UPLC. As is shown in FIG. 20, hCG was released almost completely in its intact form from hCG microspheres.

### Example 5—Synthesis of SynBiosys 20[PCL-PEG3000-PCL]-b-[PDO] Multi-Block Copolymer

[Poly(ε-caprolactone)-co-polyethylene glycol-co-poly(ε-caprolactone)]-b-[poly(L-lactide)] multi-block copolymers as used in Example 4 are known to degrade relatively slow. Multi-block copolymers composed of a poly(p-dioxanone) based crystalline block are known to degrade faster which could be beneficial in preventing polymer carrier accumulation upon repeated subcutaneous administration.

This example describes the synthesis and characterization of [poly(ε-caprolactone)-co-polyethyleneglycol-co-poly(e-caprolactone)-]b-[poly(p-dioxanone)] multi-block copolymers based on PEG3000 and a block ratio of 20/80 wt. %.

Poly(ε-caprolactone)-co-PEG3000-co-poly(ε-caprolactone) pre-polymers (abbreviated as PCL-PEG3000-PCL) with a molecular weight (Me) of around 4000 g/mol were prepared by ring-opening polymerization of ε-caprolactone using polyethyleneglycol with a molecular weight of 3000 g/mol (PEG3000) as initiator and stannous octanoate as a catalyst. Poly(p-dioxanone) pre-polymers (abbreviated as PDO) with a molecular weight (Me) of around 2500 g/mol were synthesized by ring-opening polymerization of p-dioxanone using 1,4-butanediol as initiator and stannous octanoate as a catalyst. Molecular weights of the pre-polymers were analyzed by ¹H-NMR.

[PCL-PEG3000-PCL]-b-[PDO] multi-block copolymers with a block ratio of 20/80 wt. % abbreviated as 20[PCL-PEG3000-PCL]-b-[PDO] were prepared by chain-extension of PCL-PEG3000-PCL pre-polymer with PDO pre-polymer in p-dioxane using 1,4-butanediisocyanate as a chain extender followed by freeze-drying or precipitation to remove p-dioxane.

The polymers were analysed for polymer composition by ¹H-NMR, intrinsic viscosity (Ubbelohde, chloroform), residual p-dioxane content (gas chromatography) and thermal characteristics by modulated differential scanning calorimetry. Table 10 lists the characteristics of the various [poly(ε-caprolactone)-co-PEG-co-poly(ε-caprolactone)]-b-[poly(p-dioxanone)] multi-block copolymers.

TABLE 10
Characteristcis of 20[PCL-PEG3000-PCL]-b-[PDO] multi-block
copolymers: poly(p-dioxanone) block length, intrinsic viscosity
and thermal characteristics (melting temperature Tm and
melting enthalpy ΔH_{m.}

| | Mn poly(p-dioxanone) | IV | T. | ΔHₘ |
|---|---|---|---|---|
| RCP | block (g/mol) | (dl/g) | (° C.) | (J/g) |
| 1557 | 2259 | 0.79 | 83 | 61 |
| 1568 | 2378 | 0.70 | 97 | 94 |
| 1801 | 2618 | 0.79 | 87 | 48 |
| 1803 | 2683 | 0.69 | 90 | 115 |
| 1811 | 2594 | 0.70 | 88 | 102 |
| 1814 | 2666 | 0.69 | 90 | 100 |

### Example 6—Production of hCG Extended Release Microspheres Composed of SynBiosys 20[PCL-PEG3000-PCL]-b-[PDO] Multi-Block Copolymer

hCG (Dong-A Pharmaceutical Co., Ltd. Korea) was concentrated to 30 mg/ml as described in Example 4. 1.5 g of 20[PCL-PEG3000-PCL]-b-[PDO] multi-block copolymer (RCP-1557) was dissolved in dichloromethane to a concentration of 15 wt. %. 0.73 g of the concentrated hCG solution was added to the polymer solution and homogenized at 22 000 rpm for 40 seconds to yield a water-in-oil (W/O) primary emulsion. The primary emulsion was then emulsified with a 4.0% aqueous PVA solution containing 5.0 w/v % NaCl via membrane emulsification using a membrane with 20 μm pores thereby forming a water-in-oil-in-water (W/O/W) double emulsion. The W/O/W emulsion was stirred for 3 hours at room temperature to allow extraction and evaporation of dichloromethane. After completion of solvent evaporation, the hCG microspheres were collected by filtration and lyophilized to yield dry hCG microspheres.

The hCG microparticles, characterized using the methods described in Example 4, were spherical with a smooth surface morphology (FIG. 21) and had an average particle size of 38 μm and a narrow particle size distribution (CV=14-18%). The hCG content varied from 1.33 to 1.62 wt. % representing encapsulation efficiencies of 67 to 86% (Table 10). All microparticles released rhCG gradually and mainly intact over a period of 11 weeks (FIG. 22) without any significant burst release.

TABLE 11
Overview of hCG microspheres made
of [PCL-PEG3000-
PCI]-b-[PDO]

| | Size distribution | | | |
|---|---|---|---|---|
| Batch nr | D50 (μm) | CV (%) | hCG (wt.%) | EE (%) |
| JA16085 | 38 | 18% | 1.62 | 86.0 |
| JA16101 | 38 | 14% | 1.33 | 68.0 |
| JA16102 | 39 | 17% | 1.33 | 67.4 |

Batches JA16101 and JA16102 were combined into one batch for further testing of in vivo pharmacokinetics/pharmacodynamics.

### Example 7—Pilot Pharmacokinetic/Pharmacodynamics Study of hCG Extended Release Microspheres in Young Adult Cynomolgus Monkeys

An in vivo pharmacokinetics/pharmacodynamics study with hCG extended release microspheres collected from JA16101 and JA16102 (Example 5) was conducted in five healthy, young adult male Cynomolgus monkeys.

For the duration of the study, all monkeys were treated with a GnRH antagonist (Cetrorelix 250 μg) every three days in order to suppress pituitary function and endogenous testosterone production. All monkeys were pre-treated with Cetrorelix (days −5 and −2), and assessed for both hCG and testosterone levels. Two monkeys received daily subcutaneous injections of 3 μg of hCG for the duration of the study (Control group): one monkey was dosed with Ovidrel and the other monkey was dosed with Dong-A hCG. Three monkeys were administered subcutaneously a single dose of hCG extended release microspheres (200 μg, 600 μg, and 1200 μg of r-hCG) (hCG-MSP Group).

For both groups, blood samples were obtained frequently over the first 24 hours and at regular intervals thereafter. The resulting serum was assessed for hCG by an ELISA method (LLOQ=0.5 ng/ml) and for testosterone levels by a LC/MS/MS method (LLOQ=0.25 ng/ml) until hCG and testosterone levels dropped to low levels.

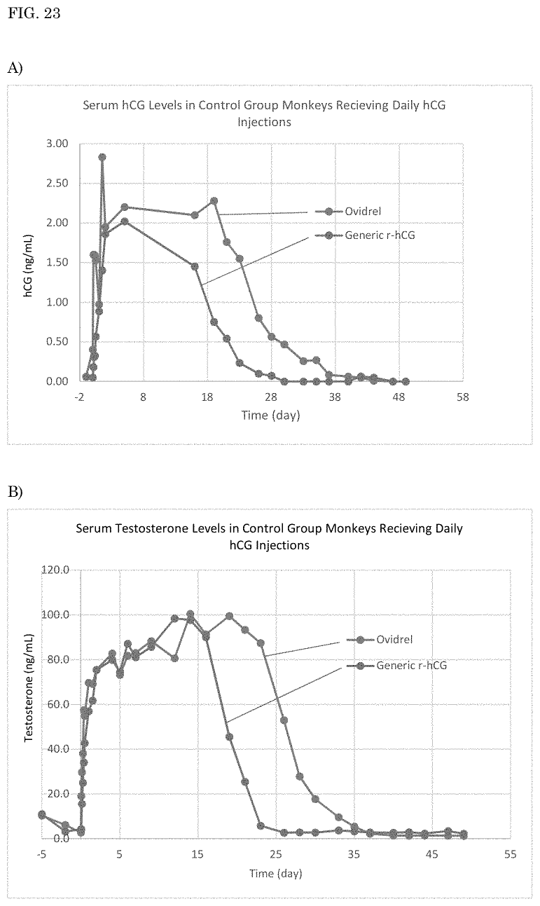

Monkeys in the control group initially had a rise in serum hCG levels with a corresponding rise in serum testosterone levels (FIG. 23). Serum levels reached a steady state by day 3. However, both serum hCG and serum testosterone levels later declined ultimately to nearly zero, despite continued daily injections of hCG.

A binding inhibition analysis was used to confirm the presence of anti-drug antibodies (ADA) against hCG. Spike recovery analysis on samples collected on day 55 from monkeys in the control group confirmed that the decline in serum hCG and serum testosterone levels was associated with an ADA response to hCG. Spiking of both the 5 and 55 ng/nl reference standards to treated monkey serum led to complete inhibition of hCG recovery using the ELISA assay as opposed to more than 80%+recovery when the naive monkey serum or pre-dose was spiked.

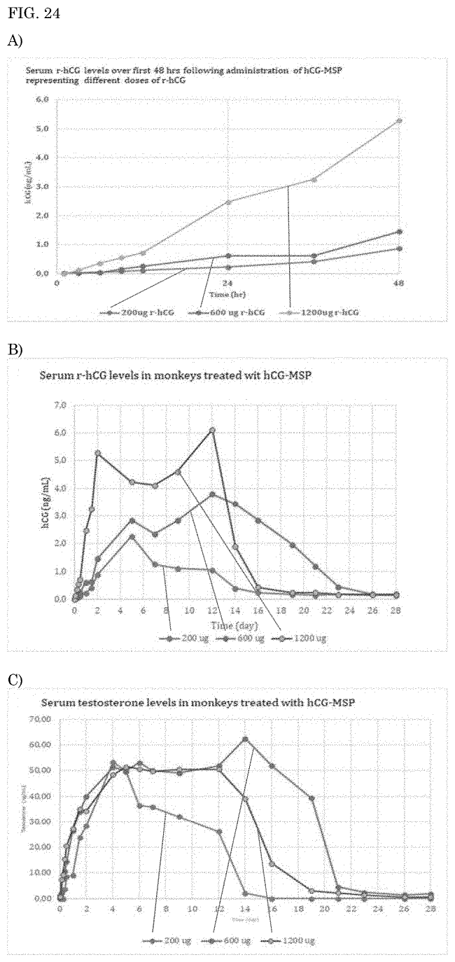

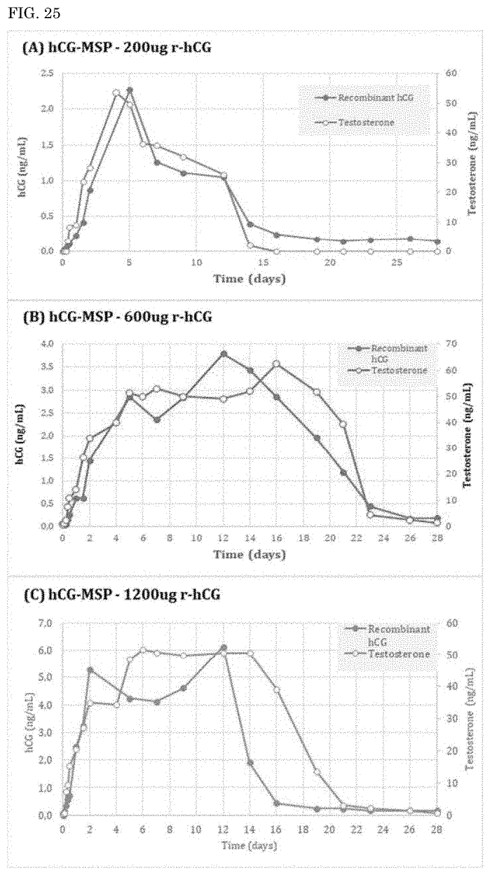

For all three monkeys treated with hCG extended release microspheres, hCG levels rise in a near-linear and dose dependent fashion. Serum hCG levels over the 48 hours confirmed that there was nearly zero burst, even for the highest dose (FIG. 24, panel A). Serum hCG levels and serum testosterone levels over the duration of the study are graphed in FIG. 24 panel B and panel C). FIG. 25 shows serum hCG and serum testosterone on the same graph for each individual monkey receiving hCG extended release microspheres representing 200 μg (panel A), 600 μg (panel B) and 1200 μg hCG (panel C). These graphs show clear parallel between serum hCG and serum testosterone levels throughout the study. Additionally, they show that serum hCG is released in a steady fashion until a significant decrease occurs around 14 days. Spike recovery analysis of hCG extended release microspheres treated monkey serum collected on day 33 confirmed that the decline in serum hCG levels was caused by an inactivating ADA response, similar to the control group.

TABLE 12
Mean recovery of hCG after spiking of hCG (Ovidrel) into
monkey serum. Suppression of recovered hCG indicates ADA
formation

| | Concentration | Recovery | Recovery |
|---|---|---|---|
| | of | from serum | from serum |
| | Ovidrel | before r-hCG | after |
| | Added | exposure | r-hCG |
| Animal | (ng/ml) | (ng/ml) | exposure (ng/ml) |
| Control #1 | 0.0 | | 0.2 |
| | 5.0 | 4.1 | 0.1 |
| | 55.0 | | 0.5 |
| Control #2 | 0.0 | | 0.1 |
| | 5.0 | 3.9 | 0.1 |
| | 55.0 | | 0.1 |
| hCG | 0.0 | | 0.0 |
| microspheres | 5.0 | 4.5 | 0.2 |
| 200 ug hCG | 55.0 | | 1.3 |
| hCG | 0.0 | | 0.0 |
| microspheres | 5.0 | 4.0 | 0.5 |
| 600 ug hCG | 55.0 | | 3.0 |
| hCG | 0.0 | | 0.0 |
| microspheres | 5.0 | 3.3 | 0.2 |
| 1200 ug hCG | 55.0 | | 1.3 |

In conclusion, this pilot study demonstrates that a single subcutaneous injection of hCG extended release microspheres provides for dose-dependent, sustained release of hCG in Cynomolgus monkeys with minimal burst and that hCG released from the microspheres retains its biologic activity and induces a testosterone response.

However, due to limitations of the primate model related to formation of anti-drug antibodies (ADA) to the recombinant human protein, pharmacokinetics over the full duration of release could not be evaluated.

As anti-drug antibodies are known to form against hCG in animals after repeated exposure, the formation of ADAs against hCG in this study was not surprising. Overall these observations indicate that the drop off in hCG levels does not reflect a problem with the formulation but is rather simply a limitation of animal models for evaluation of sustained release of a human protein. Development of inactivating ADAs is not expected in the human and is not likely to diminish the therapeutic effect. Human males have very low, but detectable, naturally occurring levels of hCG in adulthood and would not be expected to have an immune response to administration of hCG. In conclusion, a single subcutaneous injection of hCG extended release microspheres provides for dose-dependent, sustained release of hCG in Cynomolgus monkeys with minimal burst.

### Example 8—Ovidrel-Based hCG Microspheres Composed of SynBiosys Multi-Block Copolymer 20[PCL-PEG3000-PCL]b-[PDO]

This example describes the preparation and characterization of hCG extended release microspheres using Ovidrel as an alternative hGC source. Different batches of 20[PCL-PEG3000-PCL]b-[PDO] (RCP-1801, RCP-1803, RC1811 and RCP-1814) synthesized as described in Example 5 were used.

hCG solution (Ovidrel, Serono) was concentrated to 30 mg/ml as described in Example 4. The integrity of hCG was maintained during the concentration step. SEC-UPLC analysis confirmed the absence of aggregates and hCG degradation. hCG extended release microspheres were manufactured at a scale of 1.5 g according to the general procedure described in Example 5 while varying critical formulation and process parameters (Table 13).

All hCG microspheres were characterized for particle size distribution by laser diffraction. The particles had a narrow particle size distribution with an average particle size of 38 to 49 μm (Table 14). Microscopic examination by SEM showed that all microparticles had a smooth surface morphology.

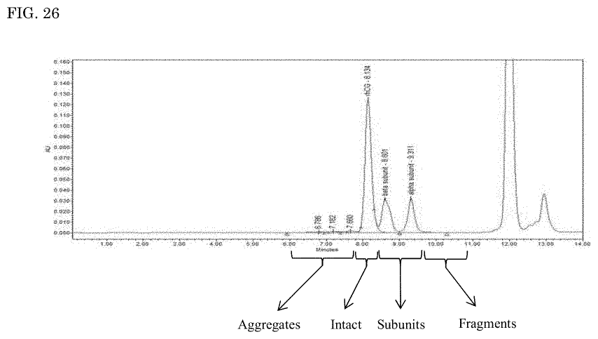

The hCG UPLC method was optimized for maximum resolution between intact hCG and degradation products, mainly consisting of its subunits. This method was performed on a Waters Acquity H-Class UPLC system, equipped with a photodiode array (PDA) detector and a fluorescence detector. hCG integrity was determined by comparing the concentration of intact protein with the total concentration of all hCG related compounds. An example of a typical chromatogram containing intact hCG, α- and β subunits, soluble aggregates and protein fragment is shown in FIG. 26.

TABLE 13
Formulation and process parameter settings used for preparation
of Ovidrel-based hCG extended release microspheres.

| | | Pol. conc. | hCG conc. | Primary | PVA conc. | CP/DP |
|---|---|---|---|---|---|---|
| Batch | RCP | (wt. %) | (mg/ml) | emulsification | (w/v %) | ratio |
| SR18-031 | 1801 | 15.0 | 28.1 | 22k rpm, 40 sec | 4.0 % | 150 |
| MS18-035 | 1803 | 12.5 | 31.2 | 22k rpm, 40 sec | 4.0 % | 150 |
| MS18-036 | 1803 | 17.5 | 30.6 | 22k rpm, 40 sec | 4.0 % | 150 |
| MS18-037 | 1803 | 15.0 | 27.2 | 22k rpm, 40 sec | 4.0 % | 150 |
| MS18-038 | 1803 | 15.0 | 31.7 | 22k rpm, 40 sec | 4.0 % | 150 |
| MS18-039 | 1803 | 15.0 | 29.9 | 22k rpm, 40 sec | 4.0 % | 125 |
| MS18-041 | 1803 | 15.0 | 28.3 | 22k rpm, 40 sec | 2% | 150 |
| MS18-042 | 1803 | 15.0 | 29.9 | 22k rpm, 40 sec | 3% | 150 |
| MS18-043 | 1803 | 15.0 | 30.3 | 18k rpm; 40 sec | 4.0% | 150 |
| M518-044 | 1803 | 15.0 | 29.1 | 22k rpm; 20 sec | 4.0% | 150 |
| M518-048 | 1811 | 15.0 | 28,6 | 22k rpm; 40 sec | 4.0% | 150 |
| M518-049 | 1814 | 15.0 | 30,6 | 22k rpm; 40 sec | 4.0% | 150 |
| M518-050 | 1803 | 15.0 | 28.5 | 22k rpm, 40 sec | 4.0% | 137.5 |

TABLE 14
Average particle size, hCG content, encapsulation efficiency and
hCG integrity of Ovidrel hCG extended release microparticles.

| Batch | d₅₀ (μm) | hCG content (wt. %) | EE (%) | rhCG integr. (%) |
|---|---|---|---|---|
| SR18-031 | 49 | 1.24 | 66 | 100 |
| MS18-035 | 42 | 0.88 | 44 | 100 |
| MS18-036 | 38 | 1.73 | 87 | 99 |
| MS18-037 | 38 | 1.82 | 91 | 100 |
| MS18-038 | 40 | 1.79 | 89 | 99 |
| MS18-039 | 39 | 1.93 | 97 | 100 |
| MS18-041 | 41 | 1.38 | 69 | 100 |
| MS18-042 | 40 | 1.54 | 77 | 100 |
| MS18-043 | 39 | 1.36 | 68 | 100 |
| MS18-044 | 40 | 1.81 | 91 | 100 |
| MS18-048 | 41 | 1.25 | 62 | 100 |
| MS18-049 | 40 | 1.13 | 57 | 100 |
| MS18-050 | 39 | 1.59 | 79 | 100 |

hCG contents as determined by extraction of hCG from the microparticles and analysis of the hCG concentration by the optimized SEC-UPLC method varied from 0.88% (EE 44%) to 1.93% (EE 97%) (Table 14).

The in vitro release kinetics were analyzed using an optimized method with higher buffer capacity that allowed more accurate identification and quantification of hCG and hCG integrity. hCG microspheres were incubated in vials containing 1.0 ml 100 mM phosphate buffer pH 7.4 containing 0.025% Tween-20 and 0.02% sodium azide and placed in a shaking thermostatic water bath at 37° C. Samples were taken twice a week with the restriction of a two days sampling interval within a week. At each sampling time point, the samples were centrifuged and 0.85 ml of the supernatant was removed for analysis. Samples were washed twice with fresh IVR-medium and the removed volume was replaced with fresh PBS buffer. Total and intact hCG content in in vitro release samples were determined with SEC-UPLC. The integrity of released rhCG was established only for samples that were taken after a sampling interval of two days, to assure minimum degradation of released rhCG (in solution).

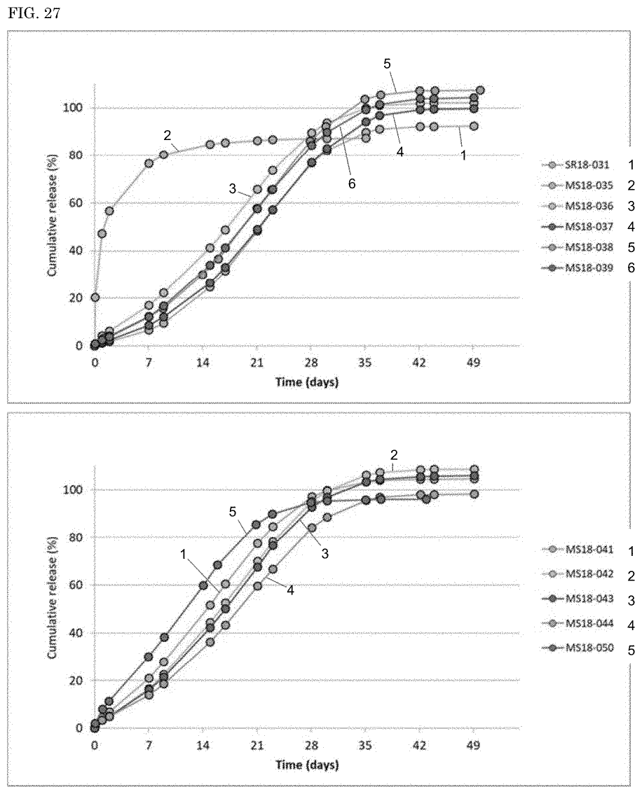

Using the optimized in vitro release assay, hCG released significantly faster from hCG extended release microparticles as compared to the old method. FIG. 27 shows cumulative release kinetics of 20[PCL-PEG3000-PCL]b-[PDO]-based hCG extended release microspheres prepared according to Table 13. All formulations, except for MS18-035 which was prepared using of low polymer concentration of 12.5% showed similar hCG in vitro release kinetics characterized by a low burst release followed by almost linear release of rhCG between 1 and 4 weeks, and a total duration of release of approximately 5 weeks. The integrity of released rhCG as measured by SEC-UPLC varied between 85 to 99% (Table 15).

TABLE 15
Integrity of released rhCG.

| | Integrity of released rhCG (%) | | | | | | | | | | |
|---|---|---|---|---|---|---|---|---|---|---|---|
| Time | SR18- | MS18- | MS18- | MS18- | MS18- | MS18- | MS18- | MS18- | MS18- | MS18- | MS18- |
| (days) | 031 | 035 | 036 | 043 | 044 | 042 | 041 | 050 | 039 | 037 | 038 |
| 0.1 | 96.4 | 98.5 | 97.1 | 97.6 | 97.8 | 96.7 | 97.2 | 97.8 | 97.4 | 97.2 | 97.3 |
| 16.9 | 89.9 | 88.6 | 91.7 | 91.3 | 90.8 | 91.2 | 91.4 | 94.3 | 90.7 | 89.6 | 91.8 |
| 23.1 | 94.0 | 93.1 | 94.0 | 94.2 | 93.9 | 93.0 | 92.6 | 95.6 | 93.3 | 93.9 | 96.3 |
| 43.9 | 97.6 | — | 92.6 | 91.2 | 95.9 | 96.5 | — | 85.7 | 94.1 | 92.9 | 94.9 |

Overall, it can be concluded that the microencapsulation process is robust and reproducible yielding hCG extended release microspheres with a narrow particle size distribution, an average hCG content of −1.62 wt. %, an acceptable EE of >80%, good integrity of the encapsulated hCG (>86%) and sigmoidal in vitro release profile with a duration of −5 weeks.

### Example 9—Bioactivity of Encapsulated hCG and hCG Released from hCG-MSP

The bioactivity of encapsulated and released hCG was measured in a mouse MA-10 Leydig cell bioassay. Test samples were generated from three representative lots of 20[PCL-PEG3000-PCL]b-[PDO]-based hCG extended release microspheres (MS18-031, MS18-037 and MS18-038) prepared as described in Example 8. The extended release microspheres were subjected to an in vitro release assay whereby hCG was released and collected after 2 hours, 23 days and 37 days. In addition, in order to evaluate the stability of encapsulated rhCG, hCG was extracted from a lot of extended release microspheres that had been stored frozen for 5 months and tested in the Leydig cell bioassay.

The bioactivity of the released rhCG from each hCG-MSP lot was measured and compared to the bioactivity of Ovidrel (recombinant Chorionic Gonadotropin) using the hCG bioassay which measures the hCG-induced production of progesterone in the murine Leydig cell tumor line, MA-10 via an ENZO progesterone enzyme linked immunosorbent assay (ELISA) kit.

The total hCG concentration and percent of intact hCG of each test sample as measured by high-performance liquid chromatography (SEC-UPLC) method is listed in Table 16.

TABLE 16
List of test samples, total hCG concentration and % intact hCG.

| | | Total | |
|---|---|---|---|
| | | hCG Conc. | % intact |
| Sample Name | Description | (ug/mL) | hCG |
| hCG Ref STD (Ovidrel) | Ovidrel Lot#BCEA | 3.36 | 100 |
| | 17062 | | |
| MS18-037 IVR 2 hr | Beginning IVR 2 hours | 1.7769 | 96.9 |
| MS18-038 IVR 2 hr | Beginning IVR 2 hours | 2.489 | 97.2 |
| SR18-031 IVR 2 hr | Beginning IVR 2 hours | 1.269 | 93.3 |
| MS18-037 IVR 23 days | Mid IVR 23 days | 31.163 | 93.1 |
| MS18-038 IVR 23 days | Mid IVR 23 days | 29.353 | 92.7 |
| SR18-031 VR 23 days | Mid IVR 23 days | 23.761 | 92.9 |
| MS18-037 IVR 37 days | End IVR 37 days | 10.806 | 91. |
| MS18-038 IVR 37 days | End IVR 37 days | 4.860 | 86.9 |
| SR18-031 IVR 37 days | End IVR 37 days | 3.172 | 85.2 |
| AMD-18-022-1 (Extr) | hCG extracted from | 58.44 | 98.8 |
| | hCG MSPs | | |
| AMD-18-022-4 (Spk) | hCG spiked in | 63.77 | 100 |
| | extraction buffer | | |

The measured levels of progesterone induced by the different hCG samples are summarized in Table 17.

TABLE 17
hCG-induced progesterone levels

| hCG | Absorbance values | | | BackCalcCouc.(pg/mL) | | | | | | Average | | | % dif- |
|---|---|---|---|---|---|---|---|---|---|---|---|---|---|
| Test Sample | Rep. 1 | Rep. 2 | Rep. 3 | Rep. 1 | Rep. 2 | Rep. 3 | X dillution factor (5) | | | (pg/mL) | StdDev | CV | ference |
| Ovidrel | 9.1878 | 0.1917 | 0.2 | 94.229 | 91.442 | 85.758 | 471.145 | 457.210 | 428.790 | 452-38 | 21.59 | 4.8% | |
| MS18-037 IVR | 0.1995 | 0.2063 | 0.2414 | 86.091 | 81.647 | 61.228 | 430.455 | 498.235 | 306.140 | 381.61 | 66.30 | 17.4% | −15.6% |
| 2 hr | | | | | | | | | | | | | |
| MS18-038 IVR | 0.248 | 0.2006 | 0.2079 | 82.611 | 85.359 | 80.629 | 413.055 | 426.795 | 403.145 | 414.33 | 11.88 | 2.9% | −8.4% |
| 2 hr | | | | | | | | | | | | | |
| SR18-031 IVR | 0.214 | 0.1952 | 0.1849 | 76.823 | 89.005 | 96.354 | 384.175 | 445.025 | 481.770 | 436.99 | 49.29 | 11.3% | −3.4% |
| 2 hr | | | | | | | | | | | | | |
| MS18-037 IVR 23 | 0.212 | 0.2094 | 0.2094 | 78.064 | 79.683 | 79.683 | 390.320 | 395.415 | 398.415 | 395.72 | 4.67 | 1.2% | −12.5% |
| days | | | | | | | | | | | | | |
| MS18-038 IVR 23 | 0.2094 | 0.2069 | 0.2195 | 79.683 | 81.264 | 73.526 | 398.415 | 406.320 | 367.630 | 396.79 | 20.44 | 5.2% | −13.6% |
| days | | | | | | | | | | | | | |
| SR18-031 IVR 23 | 0.2262 | 0.2352 | 0.2221 | 69.624 | 64.583 | 71.995 | 348.120 | 322.915 | 359.975 | 343.67 | 18.93 | 5.5% | −24.0% |
| days | | | | | | | | | | | | | |
| MS18-037 IVR 37 | 0.2143 | 0.2075 | 0.2024 | 76.651 | 80.882 | 84.172 | 383.255 | 404.410 | 420.860 | 402.84 | 18.85 | 4.7% | −11.0% |
| days | | | | | | | | | | | | | |
| MS18-038 IVR 37 | 0.2542 | 0.2382 | 0.246 | 54.556 | 62.949 | 58.793 | 272.780 | 314.745 | 293.965 | 293.83 | 20.98 | 7.1% | −35.0% |
| days | | | | | | | | | | | | | |
| SR18-031 IVR 37 | 0.2212 | 0.2246 | 0.2282 | 72.523 | 70.544 | 68.485 | 362.615 | 352.720 | 342.425 | 352.59 | 10.10 | 2.9% | −22.1% |
| days | | | | | | | | | | | | | |
| AMD-18-022-1 | 0.1996 | 0.2059 | 0.2102 | 86.024 | 81.903 | 79.182 | 430.120 | 409.515 | 395.910 | 411.85 | 17.22 | 4.2% | −9.0% |
| (Extr) | | | | | | | | | | | | | |
| AMD-18-022-4 | 0.1994 | 0.1989 | 0.2011 | 86.158 | 86.493 | 85.028 | 430.796 | 432.465 | 425.140 | 429.47 | 3.84 | 0.9% | −5.1% |
| (Spk) | | | | | | | | | | | | | |

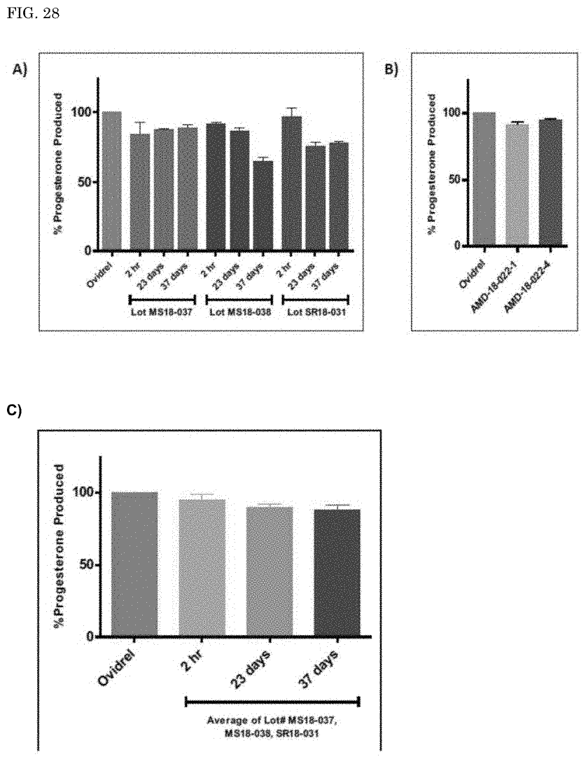

The percent difference in the ability of the hCG test samples to induce progesterone production in MA-10 cells was calculated relative to the reference standard, see Table 18 and FIG. 28 (panel A and B).

TABLE 18
hCG-induced progesteron levels as a percentage of Ovidrel-induced
progesterone. For comparison, the intact percentage previously
determined by HPLC-SEC is presented for the 9 IVR samples.

| | | | | | | Intact % for |
|---|---|---|---|---|---|---|
| | | | | | | IVR samples |
| | % Preogesterone Produced by Leydig Cell BioAssay | | | | | (Previously |
| hCG Test Sample | Rep. 1 | Rep. 2 | Rep. 3 | Std Dev | Average | Reported) |
| Ovidrel | 100 | 100 | 100 | | 100 | |
| MS18-037 IVR 2 hours | 95.15 | 90.24 | 67.67 | 0.147 | 84.36 | 96.9 |
| MS18-037 IVR 23 days | 86.28 | 88.07 | 88.07 | 0.010 | 87.47 | 93.1 |
| MS18-037 IVR 37 days | 84.72 | 89.40 | 93.03 | 0.042 | 89.05 | 91.5 |
| MS18-038 IVR 2 hours | 91.31 | 94.34 | 89.12 | 0.026 | 91.59 | 97.2 |
| MS18-038 IVR 23 days | 88.07 | 89.82 | 81.27 | 0.045 | 86.38 | 92.7 |
| MS18-038 IVR 37 days | 60.30 | 69.58 | 64.98 | 0.046 | 64.95 | 86.9 |
| SR18-031 IVR 2 hours | 84.92 | 98.37 | 106.5 | 0.109 | 96.60 | 93.3 |
| SR18-031 IVR 23 days | 76.95 | 71.38 | 79.57 | 0.042 | 75.97 | 92.9 |
| SR18-031 IVR 37 days | 80.16 | 77.97 | 75.69 | 0.022 | 77.94 | 85.2 |
| AMD-18-022-1(Extr) | 95.08 | 90.52 | 87.52 | 0.038 | 91.04 | |
| AMD-18-022-4(Spk) | 95.23 | 95.60 | 93.98 | 0.0085 | 94.93 | |

For the IVR samples, the average of the three lots at each sampling time was calculated (Table 19 and plotted in FIG. 28 (panel C).

TABLE 19
Average percent progesterone produced by hCG released from lots
MS18-037, MS18-038, SR18-031 at 2 hr, 23 days and 37 days, relative
to Ovidrel.

| | Progesterone Conc. (pg/mL) | | | | % |
|---|---|---|---|---|---|
| | Rep.1 | Rep.2 | Rep.3 | Average | Progesterone |
| Ovidrel | 471.145 | 457.21 | 428.79 | 452.3817 | 100 |
| MS18-037 | 430.455 | 408.235 | 306.14 | 410.9772 | 90.85 |
| IVR | | | | | |
| 2 hours | | | | | |
| MS18-038 | 413.055 | 426.795 | 403.145 | | |
| IVR | | | | | |
| 2 hours | | | | | |
| SR18-031 | 384.175 | 445.025 | 481.77 | | |
| IVR | | | | | |
| 2 hours | | | | | |
| MS18-037 | 390.32 | 398.415 | 398.415 | 376.725 | 83.28 |
| IVR | | | | | |
| 23 days | | | | | |
| MS18-038 | 398.415 | 406.32 | 367.63 | | |
| IVR | | | | | |
| 23 days | | | | | |
| SR18-031 | 348.12 | 322.915 | 359.975 | | |
| IVR | | | | | |
| 23 days | | | | | |
| MS18-037 | 383.255 | 404.410 | 420.860 | 349.753 | 77.31 |
| IVR | | | | | |
| 37 days | | | | | |
| MS18-038 | 272.78 | 314.745 | 293.965 | | |
| IVR | | | | | |
| 37 days | | | | | |
| SR18-031 | 362.615 | 352.72 | 342.425 | | |
| IVR | | | | | |
| 37 days | | | | | |

The data shows that hCG extracted from or released from microspheres is able to induce the production of progesterone by murine MA-10 Leydig cells. For extracted hCG, the biological activity was similar to that of the reference standard (>90%) suggesting that the Ovidrel in hCG-MSP maintained its potency when stored frozen for 5.5 months. Furthermore, the extraction solvent did not compromise the hCG integrity nor did it interfere with the hCG bioassay. For hCG released from the hCG-MSP, the progesterone response was maintained over early, mid and late release time points. Progesterone responses were above 75% in 8 out of 9 release samples and above 84% in 6 of 9 samples. While there was a decrease in progesterone response over time, this did not occur for all hCG-microsphere batches, where for example, batch MS18-037 demonstrated no change in activity over the 3 time points.

Overall, this study demonstrates that hCG can be encapsulated in microspheres and released over time from hCG extended release microspheres whilst maintaining a pharmacologically significant portion of its biological activity.

### Example—10 2-AB Glycan Mapping of Microencapsulated hCG

hCG is a highly glycosylated and sialylated molecule. hCG sialylation is a CQA affecting receptor interaction, transduction signaling, pharmacokinetics and in vivo exposure. The linkers of the sugar moieties to the hCG molecule, particularly in relation to terminal sialic acid, are potentially labile. To investigate whether the encapsulation of hCG into the microspheres via the water-in-oil-in-water process may have impacted sialylation levels or general glycosylation, which may in turn influence the pharmacokinetics without necessarily changing in vitro bioactivity of the protein, the sialylation and general glycosylation levels of microencapsulated hCG were analyzed by characterization 2-AB glycan mapping. 20[PCL-PEG3000-PCL]b-[PDO]-based hCG-MSP (MS18-037) were prepared as described in Example 8. hCG was extracted according to the procedure described in Example 7 and the concentration and integrity of extracted of hCG were determined by SEC-UPLC (Table 20). To take into account possible matrix interference related to extraction buffer (i.e., PBS with 0.2% SDS: MeOH (67:33)) or potential effects of vacuum concentration required for sample preparation, several layers of controls were included.

TABLE 20
Samples characterized by 2-AB glycan mapping

| | | | | hCG | |
|---|---|---|---|---|---|
| | | | | conc. | hCG |
| # | Description | Intended use | Code | (mg/mL) | integrity (%) |
| 1 | hCG extracted from | Study effect | AMD18-023-1B | 1.00 | 99.9 |
| | hCG-MSP | microparticle manuf. | AMD18-023-2B | 1.00 | 98.0 |
| | | Process on | | | |
| | | glycosylation of hCG | | | |
| 2 | Carrier polymer | Study any potential | AMD18-023.3 | 0.00 | n.a. |
| | extracted in | interference of the | | | |
| | extraction buffer | carrier polymer on | | | |
| | | glycan analysis | | | |
| 3 | hCG in extraction | Study effect of | AMD18-023-4B | 0.90 | 98.8 |
| | buffer | extraction and | | | |
| | | vacuum drying on | | | |
| | | glycosylation of hCG | | | |
| 4 | Ovidrel (same lot as | Baseline | AMD18-023-5 | 1.06 | 100 |
| | used for hCG-MSP | | | | |
| | preparation | | | | |

* hCG concentration and integrity as measured by SEC-UPLC

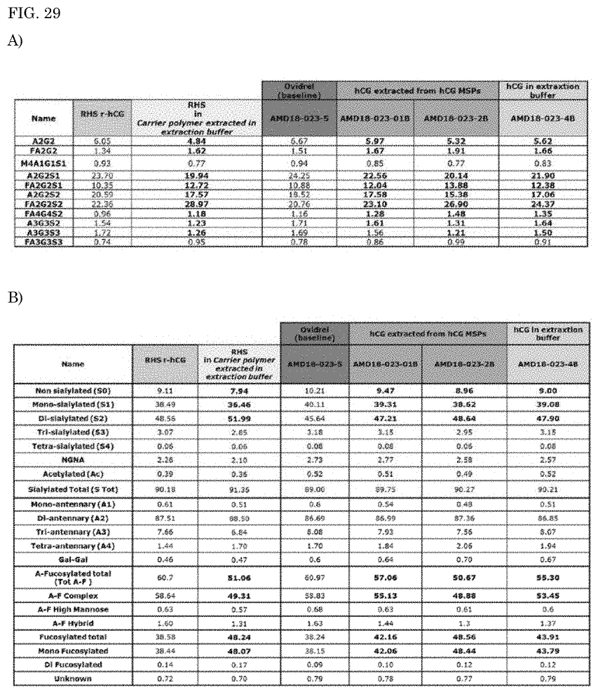

69 different glycan species were monitored for r-hCG. The abundance of each species was evaluated with respect to the total area, and expressed in terms of relative abundance. The main species are summarized in FIG. 29 panel A. All the N-glycan species were further elaborated by grouping them considering their structural features. Antennarity, galactosylation, fucosylation and sialylation distributions were obtained and are presented in FIG. 29 panel B.

In feasibility experiment with RHS, a matrix effect was observed, mainly on fucosylated and partially also on sialylated species (as shown by values for RHS vs. RHS in extraction buffer in FIG. 29, panel B)), to be considered for application of 2-AB glycan mapping from extracted material and interpretation of results. As for sialylation (that is known as a critical quality attribute for activity and efficacy of hCG), levels observed in “samples” of hCG extracted from MSPs are comparable to “relevant control in extraction buffer”, indicating no impact of encapsulation on sialylation levels vs. respective control. Differences compared to Ovidrel (“reference”, as baseline with no addition of extraction buffer) are fully aligned with the matrix effect observed in the feasibility experiment, indicating no additional interference. Together, encapsulation/extraction did not modify sialylation profile vs. relevant control. For fucosylation, some differences were present clearly related to matrix effect. A certain level of variability was observed between the two samples extracted from MSPs, to be considered for standardization of the extraction procedure if orthogonally confirmed. All other glycan species were fully aligned.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. An extended release human chorionic gonadotropin (hCG) dosage form comprising:
(a) hCG or a biologically active derivative or biologically active isoform thereof; and
(b) a biodegradable polymer microsphere,
wherein the hCG is present in the microsphere, and wherein the microsphere provides for extended release of the hCG.

2. The dosage form of claim 1, wherein the microsphere releases less than about 3% to about 40% of the hCG based on total weight of hCG present in the microsphere within about 24 hours.

3. The dosage form of claim 1, wherein the biodegradable polymer is a copolymer comprising polyethylene glycol (PEG) and poly(butylene terephthalate) (PBT).

4. The dosage form of claim 3, wherein the PEG has a molecular weight of about 1000 to about 2500 g/mol, such as about 1200 to about 2400 g/mol, about 1400 to about 2300 g/mol, about 1500 to about 2200 g/mol, about 1600 to about 2100 g/mol, about 1800 to about 2000 g/mol.

5. The dosage form of claim 3, wherein the PEG and PBT polymers are present in a weight ratio selected from the group consisting of 75/25 and 77/23, wherein the PEG comprised in the copolymer has a chain length of 1500 g/mol; and wherein the microsphere has a water polymer weight ratio between 0.5 and 1.0.

6. The dosage form of claim 1, wherein the biodegradable polymer comprises a multi-block copolymer comprising at least one hydrolysable pre-polymer (A) segment and at least one hydrolysable pre-polymer (B) segment, wherein said multi-block copolymer has a T_{g} of 37° C. or less and a Tₘ of 110-250° C. under physiological conditions, wherein the segments are linked by a multifunctional chain extender, wherein the segments are randomly distributed over the polymer chain, and wherein pre-polymer (A) segment comprises polyethylene glycol.

7. The dosage form of claim 6, wherein the pre-polymer (A) segment comprises reaction products of ester forming monomers selected from diols, dicarboxylic acids, and hydroxycarboxylic acids, preferably the pre-polymer (A) segment comprises reaction products of glycolide, lactide (D and/or L), ε-caprolactone, and/or δ-valerolactone.

8. The dosage form of claim 6, wherein the content of pre-polymer (A) in the multi-block copolymer is from about 10% to about 90% based on total weight of the multi-block copolymer, such as from about 30% to about 75%, or from about 50% to about 70%.

9. The dosage form of claim 6, wherein the pre-polymer (A) segment has a Mₙ of about 500 g/mol or more, such as about 700 g/mol or more, about 1000 g/mol or more, about 2000 g/mol or more, about 3000 g/mol or more, or about 4000 g/mol or more.

10. The dosage form of claim 6, wherein the pre-polymer (B) segment comprises poly(L-lactide), preferably poly(L-lactide) with a Mₙ of about 1000 g/mol or more, such as about 2000 g/mol or more, about 3000 g/mol or more, or about 4000 g/mol or more.

11. The dosage form of claim 6, wherein the content of pre-polymer (B) in the copolymer is about 10% to about 90% based on total weight of the multi-block copolymer, such as about 25% to about 70%, or about 30% to about 50%.

12. The dosage form of claim 6, wherein the multifunctional chain extender is a difunctional aliphatic chain extender, preferably a diisocyanate, such as 1,4-butane diisocyanate.

13. The dosage form of claim 6, wherein the polyethylene glycol has a Mₙ of about 150 to about 5000 g/mol, such as about 200 g/mol to about 1500 g/mol, about 600 to about 1000 g/mol, about 400 to about 3000 g/mol, about 600 to about 1500 g/mol, about 600 to about 5000 g/mol, or about 1000 to about 3000 g/mol.

14. The dosage form of claim 6, wherein the biodegradable multi-block copolymer has a swelling ratio under physiological conditions of about 1 to about 4, preferably about 1 to about 2, more preferably about 1 to about 1.5.

15. The dosage form of claim 6, wherein the biodegradable multi-block copolymer is a [poly(E-caprolactone)-co-polyethylene glycol-co-poly(ε-caprolactone)]-b-[poly(L-lactide)] multi-block copolymer.

16. The dosage form of claim 1, wherein the biodegradable polymer comprises a biodegradable, phase separated, thermoplastic multi-block copolymer comprising at least one amorphous hydrolysable pre-polymer (A) segment and at least one semi-crystalline hydrolysable pre-polymer (B) segment, wherein
said multi-block copolymer under physiological conditions has a T_{g} of 37° C. or less and a Tₘ of 50-110° C.;
the segments are linked by a multifunctional chain extender;
the segments are randomly distributed over the polymer chain; and
the pre-polymer (B) segment comprises a X-Y-X tri-block copolymer wherein Y is a polymerisation initiator, and X is a poly(p-dioxanone) segment with a block length expressed in p-dioxanone monomer units of 7 or more.

17. The dosage form of claim 16, wherein X is a poly(p-dioxanone) segment with a block length expressed in p-dioxanone monomer units of about 7 to about 35, such as about 8 to about 30, about 9 to about 25, about 10 to about 20, or about 12 to about 15.

18. The dosage form of claim 16, wherein at least part of the pre-polymer (A) segment is derived from a water-soluble polymer, preferably about 30% or more by total weight of pre-polymer (A) is derived from a water-soluble polymer, such as about 40 to about 95%, about 50 about 90%, or about 60 to about 85%.

19. The dosage form of claim 17, wherein about 70% or more by total weight of said pre-polymer (B) segment is poly(p-dioxanone), preferably about 80% or more, more preferably about 90% or more.

20. The dosage form of claim 16, wherein said pre-polymer (B) segment has a number average molecular weight Mₙ of about 1300 to about 7200 g/mol, preferably about 1300 to about 5000 g/mol, more preferably about 1500 to about 4500 g/mol, even more preferably about 2000 to about 4000 g/mol, most preferably about 2200 to about 3000 g/mol.

21. The dosage form of claim 16, wherein said pre-polymer (B) segment has a weight average molecular weight M_{w} of about 1800 to about 10080 g/mol, preferably about 1800 to about 7000 g/mol, more preferably about 2100 to about 6300 g/mol, even more preferably about 2600 to about 5600 g/mol, most preferably about 3000 to about 4200 g/mol.

22. The dosage form of claim 16, wherein said pre-polymer (B) has a T_{g} of less than about 0° C., preferably less than about −20° C., more preferably less than about −40° C.

23. The dosage form of claim 16, wherein said pre-polymer (B) has a Tₘ in the range of about 60 to about 100° C., preferably in the range of about 75 to about 95° C.

24. The dosage form of claim 18, wherein said water-soluble polymer is selected from the group consisting of polyethers such as polyethylene glycol (PEG), polytetramethylene oxide (PTMO), polypropyleneglycol (PPG), polyvinylalcohol (PVA), polyvinylpyrrolidone (PVP), polyvinylcaprolactam, poly(hydroxyethylmethacrylate) (poly-(HEMA)), polyphosphazenes, or copolymers of these polymers, preferably said water-soluble polymer is derived from poly(ethylene glycol) (PEG) having a Mₙ of about 150 to about 5000 g/mol.

25. The dosage form of claim 16, wherein said chain extender is a difunctional aliphatic chain-extender, preferably a diisocyanate, such as 1,4-butane diisocyanate.

26. The dosage form of claim 16, wherein pre-polymer (A) comprises reaction products of cyclic monomers and/or non cyclic monomers, wherein said non cyclic monomers are preferably selected from the group consisting of succinic acid, glutaric acid, adipic acid, sebacic acid, lactic acid, glycolic acid, hydroxybutyric acid, ethylene glycol, diethylene glycol, 1,4-butanediol and/or 1,6-hexanediol, and wherein said cyclic monomers are preferably selected from the group consisting of glycolide, lactide, ε-caprolactone, δ-valerolactone, trimethylene carbonate, tetramethylenecarbonate, 1,5-dioxepane-2-one, 1,4-dioxane-2-one (p-dioxanone) and/or cyclic anhydrides such as oxepane-2,7-dione.

27. The dosage form of claim 16, wherein the biodegradable multi-block copolymer is a [poly(c-caprolactone)-co-polyethylene glycol-co-poly(ε-caprolactone)]-b-[poly(p-dioxanone)] multi-block copolymer.

28. The dosage form of claim 16, wherein the biodegradable multi-block copolymer is represented by [(R¹R²ₙR³)_{q}]ᵣ[(R⁴ₚR⁵R⁶ₚ)]ₛ, wherein
R¹, and R³ are each 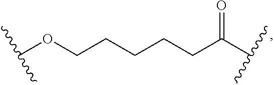 [SMILES: *OCCCCCC(*)=O]
R² is 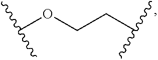 [SMILES: *CCO*]
R⁴ and R⁶ are each 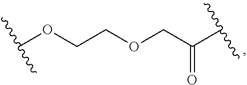 [SMILES: *OCCOCC(*)=O]
n, being the number of repeating R² moieties, is 20-115, preferably 35-100, more preferably 45-85;
p, being the number of repeating R⁴ and R⁶ moieties is 7 or more, preferably 7-35, such as 10-20, or 10-14;
q, being the number average molecular weight of the (R¹R²ₙR³) block is 1000-7000 g/mol, preferably 3000-5000 g/mol, more preferably 3800-4200 g/mol;
r/s, being the ratio of pre-polymer (A) segment over pre-polymer (B) segment is 0.10-1.0, such as 0.15-0.50, or 0.20-0.30.

29. The dosage form of claim 28, wherein n is 63-73, p is 10-14, q is 3800-4200, and r/s is 0.15-0.35.

30. The dosage form of claim 1, wherein the microsphere has a release profile that allows for extended release for between about 14 to about 50 days.

31. The dosage form of claim 1, further comprising at least one pharmaceutically acceptable excipient.

32. A method of administering the dosage form of claim 1, wherein the microsphere releases less than about 20%, preferably less than about 10%, more preferably less than about 5% of hCG based on total amount of hCG content of the microspheres within about the first 24 hours.

33. The method of claim 32, wherein the administration is intradermal, intramuscular, transdermal, or subcutaneous.

34. A method of treating a subject in need of hCG comprising administering the dosage form of claim 1.

35. The method of claim 34, wherein the treatment is for an indication selected from the group consisting of hyogonadotropism, cryptorchidism, luteal phase maintenance, contraception, pituitary gland disorders, breast cancer, and weight loss.

36. The dosage form of claim 1 for use in treating a subject in need of hCG, comprising administering the dosage form to said subject.

37. Use of a biodegradable polymer microsphere, preferably as defined in claim 1 for extended release of hCG.
